SEQUENCE LISTING (58 sequences)

SEQ ID NO: 1 - DNA, 26 bp, Artificial
  Primer sequence
gannnngcnggnganggnacnacnac

SEQ ID NO: 2 - DNA, 26 bp, Artificial
  Primer sequence
nnnnnntcnccnaanccnggngcntt

SEQ ID NO: 3 - DNA, 21 bp, Artificial
  Primer sequence
tgcaggagcaaatccaatgat

SEQ ID NO: 4 - DNA, 20 bp, Artificial
  Primer sequence
gcatggcttcggcaattaaa

SEQ ID NO: 5 - DNA, 19 bp, Artificial
  Primer sequence
gctgttgatgtagcagttg

SEQ ID NO: 6 - DNA, 20 bp, Artificial
  Probe sequence
tgttgctgcgggcatgaacc

SEQ ID NO: 7 - DNA, 20 bp, Artificial
  Primer sequence
ataaccccttcgtttcctac

SEQ ID NO: 8 - DNA, 20 bp, Artificial
  Primer sequence
aacttgacgtggttgaaggg

SEQ ID NO: 9 - DNA, 23 bp, Artificial
  Primer sequence
gttttcaagacttcttcaagcaa

SEQ ID NO: 10 - DNA, 26 bp, Artificial
  Primer sequence
gttgtcgtaccntcaccagcaatttc

SEQ ID NO: 11 - DNA, 26 bp, Artificial
  Primer sequence
aangcgcctggtttnggtgatnnnnn

SEQ ID NO: 12 - DNA, 552 bp, Unknown
  Uncultured human vaginal bacterium
gctaccgtgttggctcaatctctggtgcacgaaggcttaaagaatgttgctgctggtagcaacccgatcgctcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 13 - DNA, 558 bp, Unknown
  Uncultured human vaginal bacterium
gctacggtactagctcagagcatcattaacgtaggtctgaagaacattacctctggcgctaatcctatggaggtaaagcgtggtatcgacaaggctgtcaaggctgtcgtcgagagtatccgcaagcaagctaaggaggtgggtgatgacctagagcagatcgctcatgtggctaccatctcggctaatggcgatgaggagatcggccaactgatcgctcaggctatggagaaggtcaagaaggagggtgtcatcaccgttgaggaggccaagggtatcgatacgacggtagacattgtcgagggtatgcagtttgacaacggctatatctctccctacttcgtcactgatcctgagaagatggagtgccgtatggagaagccttacatcctcttctacgagaagaagctctctaccatcaaggatcttctaccactactagagaaggtgctccagcagggtcgctctctgctgatcattgctgaggatattgagagcgaggcattgaccacgcttgtcctcaacagactacgcgctggtcttaagatctgtggtgtc

SEQ ID NO: 14 - DNA, 552 bp, Unknown
  Uncultured human vaginal bacterium
gcaacacttcttgctcaagcaataattagagaaggacttaagaatatcgctgctggtgcaaacccaatgatattacaaaacggtatcagaaaagctgttgaaaaaactgttgaaagcatcaaatcatattcaaagaaagttgaatcatctgatgctatcgctcaagttgcttcaatttcagctggagacaaggaaataggtaagcttattgcagatgctatggataaagttggtaacgacggagttataactgttgaagaatcaaaatctatgggcacaacacttgaagttgttgaaggaatgcaatttgatagaggatacgtttcaagctacatggtaactgatacagataagatggtagctgaacttgacaatccttacatcttaatcacagataagaaaattacaaatatacaagaagttcttcctgtacttgaagaaattgttaagcaatcaaaaccattattaataatcgctgaagacattgaaggcgatgcaatggcaacacttgttgttaacaagcttagaggtacattcaattgcgttgctgtt

SEQ ID NO: 15 - DNA, 552 bp, Unknown
  Uncultured human vaginal bacterium
gctaccgtgttggctcaatctctggtgcacggaggcttaaagaatgttgttgctggtagcaacccgatcgttcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 16 - DNA, 552 bp, Unknown
  Uncultured human vaginal bacterium
gctactctacttgcacaagctatcattagagaaggtatgagaaaccttgcagcaggtgcaaacccaatggttcttcaaaagggtattgctaaggcagttgaagctgcagttgaagaaatcaaacacttctcaacagaagtttcttcaaaggaagctattgaacaagttgcttcaatttctgctgctaacgaacaagttggaaaacttatttctgaagcaatggaaaaggttggaaacgacggagttatcacagttgaagaatcaaaatctatgggaacaagcctagatgttgttgaaggtatgcaatttgacaggggctacctatctccatacatggttacagattctgacaagatggttgcagaattagaggacccatacatcctaattacagacaagaagatttcaaatatccaagaaatccttccactattagaacaagttcttcaacaatcaaaaccacttcttattattgcagatgatattgatggcgaagctcttgcaactctagttgttaactcacttaggggaacactaaatgtagttggagta

SEQ ID NO: 17 - DNA, 558 bp, Unknown
  Uncultured human vaginal bacterium
gctacggtactagctcagagcatcattaacgtaggtctgaagaacattacctctggcgctaatcctatggaggtaaagcgtggtatcgacaaggctgtcagggctgtcgtcgagagtatccgcaagcaagctaaggaggtgggtgatgacctagagcagatcgctcatgtggctaccatctcggctaatggcgatgaggagatcggccaactgatcgctcaggctatggagaaggtcaagaaggagggtgtcatcaccgttgaggaggccaagggtatcgatacgacggtagacattgtcgagggtatgcagtttgacaacggctatatctctccctacttcgtcactgatcctgagaagatggagtgccgtatggagaagccttacatcctcttctacgagaagaagctctcgaccatcaaggatctgctaccgctcctagagaaggtactccagcagggtcgctcgctactgatcatcgctgaggatatcgagagcgaggcacttacgacgctcgtcctcaacagactacgcgctggtcttaagatctgtggtgtc

SEQ ID NO: 18 - DNA, 552 bp, Unknown
  Uncultured human vaginal bacterium
gcaactgttttagcacaagcaattgttcgtgaaggtatgaagaatgttactgctggagctaatccagttggtattcgccgtggtattgaaatagctactaaggctgcagtagcagaattgcataaaattagtcatgaagtaagttctaaagctgaaatagcacaagttgcctctgtttcttcagcatcaacagaagttggtgaattgattgctgatgcaatggaacgagtaggacacgatggtgtaattacaattgaagaatctaaaggtatcgataccgaattatcagttgtagaaggtatgcaatttgatcgtggttatttatctcaatacatggttactgacaacgacaagatggaagccgatttagaaaatccatatatcttaattactgataagaagatttctaatattcaagatattttgccattattacaagaaattgtacagcaaggtaagtctttattaattattgctgatgacattgatggtgaagctttaccaactcttgttttgaacaagattcgtggcacattcaatgttgtagctgtt

SEQ ID NO: 19 - DNA, 552 bp, Unknown
  Uncultured human vaginal bacterium
gctaccgtgttggctcagtctttggtgcacgaaggcttaaagaacgttgttgccggtagcaacccaatcgctcttcgtcgtggaattgaaaaggcttcagaagcaattgttaaggaacttctcgcatctgctaaggatgttgaaaccaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 20 - DNA, 558 bp, Unknown
  Uncultured human vaginal bacterium
gctacaatattgacacaagccattgtttcagaaggtttgaaaaacgttacagctggtgctaatcctatggatttgaaaagaggaatcgacaaggcagttaatgcagtagttgattatatcaagtccaatgcagagcaagtaggcgacaattacgataagattgagcaagttgctacggtttctgctaacaatgatcctgagattggaaagcttttggctgatgccatgcgcaaagtaagcaagaatggtgtcatcactattgaggagagtaagagccgtgataccaatatcggtgttgttgaaggaatgcaattcgaccgtggatatctttccagttatttcgtaacagacacggataagatggaaagtgtcatggaaaatccatacattttgatttacgacaaaaagatttctaacatcaaagatttccttcctatcctacagccagctgcagaaagtggtcgtcctttgctcgttattgcagaagatgttgattctgaggcattgacaacattggtagtaaaccgtttgcgtgcagcattgaaaatatgtgctgtg

SEQ ID NO: 21 - DNA, 552 bp, Unknown
  Uncultured human vaginal bacterium
gctaccgtgttggctcaatctctggtgcacgaaggcttaaagaatgttgttgctggtagcaacccgatcgctcttcgtcgcggaattgaaaaggcttcagaagcaattgctaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacgacaacaagtttggtcttgatctggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 22 - DNA, 552 bp, Unknown
  Uncultured human vaginal bacterium
gctaccgtgttggctcaatctctggtgcacgaaggcttaaagaatgttgttgctggtagcaacccgatcgctcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcaagt

SEQ ID NO: 23 - DNA, 552 bp, Unknown
  Uncultured human vaginal bacterium
gctaccgtgttggctcaatctctggtgcacgaaggcttaaagaatgttgctgctggtagcaacccgatcgctcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttctgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 24 - DNA, 552 bp, Unknown
  Uncultured human vaginal bacterium
gctactctacttgcacaagctatcattagagaaggtatgagaaaccttgcagcaggtgcaaacccaatggttcttcaaaagggtattgctaaggcagttgaagctgcagttgaagaaatcaaacacttctcaacagaagtttcttcaaaggaagccattgaacaagttgcttcaatttctgctgctaacgaacaagttggaaaacttatttctgaagcaatggaaaaggttggaaacgacggagttatcacagttgaagaatcaaaatctatgggaacaagcctagatgttgttgaaggtatgcaatttgacaggggctacctatctccatacatggttacagattctgacaagatggttgcagaattagaggacccatacatcctaattacagacaagaagatttcaaatatccaagaaatccttccactattagaacaagttcttcaacaatcaaaaccacttcttattattgcagatgatattgatggcgaagctcttgcaactctagttgttaactcacttaggggaacactaaatgtagttggagta

SEQ ID NO: 25 - DNA, 552 bp, Unknown
  Uncultured human vaginal bacterium
gctaccgtgttggctcaatctctggtgcacgaaggcttaaagaatgttgttgctggtagcaacccgatcgctcttcgtcgcggaattgaaaaggcttcagaagcaattgttacggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 26 - DNA, 558 bp, Unknown
  Uncultured human vaginal bacterium
gcaacaattcttactcaatctattgttacagaaggattgaagaatgttgctgctggtgctaatcctatggatttgaagcgtggtattgataaggctgtcaataaagtggttgattttattaaggatagtgccgaaattgttggcgacaactatgataaaattgagcaagttgcaaccgtaagtgctaataatgaccctgagattggtaaacttcttgctgacgctatgcgcaaagtttctaaggacggtgttattacaattgaggaaagcaaaacacgcgacaccacaattgacgttgtagaaggtatgcaattcgaccgtggctacctttcaagttactttgttacagatactgaaaagatggaagctctgatggaaaacccatacattctcatctacgataagaaaatttctaatgttaaaggcttcttgcctatcctccaaccagctgccgaaagtggtcgtcctttgttggttattgctgaagatgttgattcagaagcattgacaacattggtcgtaaaccgtttgcgtggtggtttgaagatttgcgctgtg

SEQ ID NO: 27 - DNA, 552 bp, Unknown
  Uncultured human vaginal bacterium
gcaactgttttagcacaagcaattgttcgtgaaggtatgaagaatgttactgctggagctaatccagttggtattcgccgtggtattgaaatagctactaaggctgcagtagcagaattgcataaaattagtcatgaagtaagttctaaagctgaaatagcacaagttgcttctgtttcttcagcatcaacagaagttggtgaattgattgctgatgcaatggaacgagtaggacacgatggtgtaattacaattgaagaatctaaaggtatcgataccgaattatcagttgtagaaggtatgcaatttgatcgtggttatttatctcaatacatggttactgacaacgacaagatggaagccgatttagaaaatccatatatcttaattactgataagaagatttctaatattcaagatattttgccattattacaagaaattgtacagcaaggtaagtctttattaattattgctgatgacgttgatggtgaagctttaccaactcttgttttgaacaagattcgtggcacattcaatgttgtagctgtt

SEQ ID NO: 28 - DNA, 552 bp, Unknown
  Uncultured human vaginal bacterium
gcaacacttcttgctcaagcaataattagagaaggacttaagaatatcgctgctggtgcaaacccaatgatattacaaaacggtatcagaaaagctgttgaaaaaactgttgaaagcatcaaatcatattcaaagaaagttgaatcatctgatgctatcgctcaagttgcttcaatttcagctggagacaaggaaataggtaagcttattgcagatgctatggataaagttggtaacgacggagttataactgttgaagaatcaaaatctatgggcacaacacttgaagttgttgaaggaatgcaatttgataggggatacgtttcaagctacatggtaactgatacagataagatggtagctgaacttgacaatccttacatcttaatcacagataagaaaattacaaatatacaagaagttcttcctgtacttgaagaaattgttaagcaatcaaaaccattattaataatcgctgaagacattgaaggcgatgcaatggcaacacttgttgttaacaagcttagaggtacattcaattgcgttgctgtt

SEQ ID NO: 29 - DNA, 558 bp, Unknown
  Uncultured human vaginal bacterium
gcaacaattcttactcaatctattgttacagaaggattgaagaatgttgctgctggtgctaatcctatggatttgaagcgtggtattgataaggctgtcaataaagtggttgattttattaaggatagtgccgaaattgttggcgacaactatgataaaattgagcaagttgcaaccgtaagtgctaataatgaccctgagattggtaaacttcttgctgacgctatgcgcaaagtttctaaggacggtgttattacaattgaggaaagcaaaacacgcgacaccacaattgacgttgtagaaggtatgcaattcgaccgtggctacctttcaagttactttgttacagatactgaaaagatggaagctctgatggaaaacccatacattctcatctacgataagaaaatttctaatgttaaagacttcttgcctatcctccaaccagctgccgaaagtggtcgtcctttgttggttattgctgaagatgttgattcagaagcattgacaacattggtcgtaaaccgtttgcgtggtggtttgaagatttgcgctgtg

SEQ ID NO: 30 - DNA, 552 bp, Unknown
  Uncultured human vaginal bacterium
gctactttacttgctcaagctatcattagagaaggcatgagaaaccttgcagcaggtgcaaacccaatggttcttcaaaagggtattgctaaggcagttgaagctgcagttgaagaaatcaaacacttctcaacagaagtttcttcaaaggaagctattgaacaagttgcttcaatttctgctgctaacgaacaagttggaaaacttatttctgaagcaatggaaaaggttggaaacgacggagttatcacagttgaagaatcaaaatctatgggaacaagcctagacgttgttgaaggtatgcaatttgacagaggttacctatctccatacatggttacagattctgacaagatggttgcagaattagaagacccatacatcctaattacagacaagaagatttcaaatattcaagaaatccttccactattagaacaagttcttcaacaatcaaaaccacttcttattatcgcagatgatattgatggagaagcccttgcaactctagttgttaactcacttaggggaacacttaacgtagttggagta

SEQ ID NO: 31 - DNA, 552 bp, Unknown
  Uncultured human vaginal bacterium
gctaccgtgttggctcaatctctggtgcacgaaggcttaaagaatgttgttgctggtagcaacccgatcgctcttcgtcgcggaattgaaaaggcttcagaagcaattgttaaggaacttctcgcatctgctaaggatgttgaaacaaaggatcagattgcagctaccgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 32 - DNA, 552 bp, Unknown
  Uncultured human vaginal bacterium
gctaccgtgttggctcaatctctggtgcacgaaggcttaaagaatgttgttgctggtagcaacccgatcgctcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 33 - DNA, 552 bp, Unknown
  Uncultured human vaginal bacterium
gctaccgtgttggctcaatctctggtgcacgaaggcttaaagaatgttgttgccggtagcaacccaatcgctcttcgtcgtggaattgaaaaggcttcagaagcaattgttaaggaacttctcgcatctgctaaggatgttgaaaccaaggatcagattgcagctactgcaactatttctgcagcggatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcagta

SEQ ID NO: 34 - DNA, 552 bp, Unknown
  Uncultured human vaginal bacterium
gctaccgtgttggctcaatctctggtgcacgaaggcttaaagaatgttgttgttggtagcaacccgatcgctcttcgtcgcggaattgaaaaggcttcagaagcaattgttaaggaacttctcgcatctgctaaggatgttgaaacaaaggatcagattgcagctactgcaactatttctgcagcagatcctgaagttggcgaaaagattgctgaagctttggataaggttggtcaagacggtgttgttactgttgaagacaacaacaagtttggtcttgatttggacttcactgaaggtatgcgttttgataagggttatatttccccttacttcgtcactaatgcagaagatcagactgctgttcttgaagacccatacattttgctcacttctggcaaggtttctagccagcaagacattgttcatgttgctgaacttgttatgaagtctggtaaaccattgctcattattgctgaagatgttgatggcgaagcattaccaactcttgttcttaacaagattcgcggtactttcaatactgttgcggta

SEQ ID NO: 35 - DNA, 552 bp, Unknown
  Uncultured human vaginal bacterium
gcaactgttttgactcaagcaattactaatgaaggtatgaagaacgtaactgctggtgctaacccagttggcattcgtcgtggtattgaaaaagctactgaagctgctgttaatgaattgcacaagattagccacaaggttgaatcaagagaccaaattgctaacgtagctgcagtttcatcatcttcaaaggaagttggtaacttaatcgctgacgctatggaaaaggttggtcacgatggcgttatcactattgaagattcacgtggtatcaacactgaactttcagtagttgaaggtatgcaatttgatcgtggttacttatcacaatacatggtaactgacaacgataagatggaagcagatcttgataatccttacattttgatcactgacaagaagatctcaaacattcaagatatcttgccacttcttcaagaaattgttcaacaaggtaagtcattattgatcattgctgatgaagttactggtgaagctcttccaacacttgtattgaacaagattcgtggtaccttcaacgttgtagctgtt

SEQ ID NO: 36 - DNA, 555 bp, Unknown
  Uncultured human vaginal bacterium
gctactgtactggcatgctctattgccaaggaaggctttgagaagattagcaaaggtgctaatccagtggaaatcaggagaggtgtgatgttagctgttgatgctgtaatagctgaacctaaaaagcagtctaaacctgtgaccacccctgaagaaattgcgcaggttgctacaatttctgcaagtggagacaaagaaattggccatatcctctctgatgcagtgaaaaaggtgggaagaaagggtgtcataacagtaaaggatgggaaaacactgaatgatggattagaaattattgaaagcttgaagtttgatcgaggctatgtttctccatactttattaatacatcaaaaggtcagaaatgtgaattccaggaggcatatgttctgttgagtgaaaagaaaatttctagtgtccagtccattgtacctgctcttgaaactgccagtgctcaccgtaagcctttggtcataatcactgaagatgttggtggagaagctctaagtacacttgtcttgaacagcctaaagattgattttcaggttgtggcagtc

SEQ ID NO: 37 - DNA, 558 bp, Unknown
  Uncultured human vaginal bacterium
gcaaccatcctcacacaggctatcgttacagagggactggagaacgtcaccgctggcgcgaacccgatggacttgaagcgcggtattgacaaggctgtgaatgccgtagtggattacatcaaggcaaatgctgagctcgtaggcgacaactacgacaagatagagcaggttgccacggtgtcagcaaataacgaccctgagattggtaagctccttgctgacgctatgcgcaaggtgtcaaaggatggtgtgataaccatcgaggagagcaagagccgtgacacaagcatcggtgttgtggagggtatgcagtttgaccgtggatatctgtctggttacttcgtgacagacgcggagaagctggagtgtgttatggagaacccatatatcctcatctacgacaagaagatttcaaacctcaaggagcttcttcctatcctccagccggcggcagagagtggccgtggactgcttatcatagcagaggacgttgactctgaagcactcacaacactcgttgtaaaccgtctgcgtggcggtctgaagatatgcgctgtc

SEQ ID NO: 38 - DNA, 552 bp, Unknown
  Uncultured human vaginal bacterium
gctaccgttttgacccaagcaattgttaatgccgggatgaagaacgttacttctggtgctaacccagttggtattcgtcggggaattgacaaggctactgaagctgctgttgaagcattaaagaagatgtcccatgacgttaagactaaggatgacattgcacaaatcgcttcaatttcagctgctaacccagaagttggtaagttgattgccgatgcaatggaaaaggttggtaacgatggtgttatcactattgaagattctcgtggtgttgaaaccagtgttgacgtcgttgaagggatgagcttcgatcgcggttacatgtcacaatacatggttaccgataacgacaagatggaagctgatttggataacccatacattttgattactgacaagaagattagcaacattcaagacattttgccacttcttcaagaaattgttcaacaaggtaagtcattattgatcattgctgatgacgttactggtgaagctcttccaacacttgtattgaacaagattcgtggtaccttcaacgttgtagctgtt

SEQ ID NO: 39 - DNA, 552 bp, Unknown
  Uncultured human vaginal bacterium
gcaactgttttgactcaagcaattactaatgaaggtatgaagaacgtaactgctggtgctaacccagttggcattcgtcgtggtattgaaaaagctactgaagctgctgttaatgaattgcacaagattagccacaaggttgaatcaagagaccaaattgctaacgtagctgcagtttcatcatcttcaaaggaagttggtaacttaatcgctgacgctatggaaaaggttggtcacgatggcgttatcactattgaagattcacgtggtatcaacactgaactttcagtagttgaaggtatgcaatttgatcgtggttacttatcacaatacatggtaactgacaacgataagatggaagcagatcttgataatccttacattttgatcactgacaagaagatctctaacattcaagatatcttgccacttcttcaagaaattgttcaacaaggtaagtcattattgatcattgctgatgacgttactggtgaagctcttccaacacttgtattgaacaagattcgtggtaccttcaacgttgtagctgtt

SEQ ID NO: 40 - DNA, 552 bp, Unknown
  Uncultured human vaginal bacterium
gcaacgttacttgcacaggctttgattcgtgagggcatgaaaaacgttacggcaggtgcaaacccgatgatcctccgcaaaggtgtccagggtgctgtagatgtcgcggtaaaggcagttacggagcattccaaaaaagttagcggttccgaagatatcgcacgtgtcgcgacaatttcttctggagatgcaaccatcggcacattaattgcggaggcaatggaaaaagtgacgtcagatggtgtcatcacagtggaagaatccaaaactgcggagacatattccgaagtcgtcgaaggaatgatgtttgaccgcggatatatcacgccgtatatggttaccgatacggataagatggttgccgagattgatgacgcttatatcctgattaccgataaaaagatttccaacattcaggaaatccttccgcttctcgagaaaattgttcaggcgggtaagaaactcctgattattgcagaggatgtcgaaggtgaggcacttacgaccctgattctcaataaactgcgcggtacgtttacctgcgttgcggta

SEQ ID NO: 41 - DNA, 552 bp, Unknown
  Uncultured human vaginal bacterium
gctactgttttaactcaagctatcgttaatgaaggtatgaagaacgtaactgccggtgctaaccctgttggtattcgtcgtggtattgaaaaggctactaaggctgcagttgatgaattgcataagattagccacaaggtttctggccgtgacgaaattgcacaagtagctagcgtatcaagtgcttcagaagaagtaggtaacttaatcgctgacgctatggaaaaggttggtcacgatggcgttatcactattgaagattcacgtggtatcaacactgaactttcagtagttgaaggtatgcaatttgatcgtggttacttatcacaatacatggtaactgacaacgataagatggaagcagatcttgataatccttacattttgatcactgacaagaagatctcaaacattcaagatatcttgccacttcttcaagaaattgttcaacaaggtaagtcattattgatcattgctgatgacgttactggtgaagctcttccaacacttgtattgaacaagattcgtggtaccttcaacgttgtagctgtt

SEQ ID NO: 42 - DNA, 552 bp, Unknown
  Uncultured human vaginal bacterium
gcaactgttttgactcaagcaattactaatgaaggtatgaagaacgtaactgctggtgctaacccagttggcattcgtcgtggtattgaaaaagctactgaagctgctgttaatgaattgcacaagattagccacaaggttgaatcaagagaccaaattgctaacgtagctgcagtttcatcatctttaaaggaagttggtaacttaatcgctgacgctatggaaaaggttggtcacgatggcgttatcactattgaagattcacgtggtatcaacactgaactttcagtagttgaaggtatgcaatttgatcgtggttacttatcacaatacatggtaactgacaacgataagatggaagcagatcttgataatccttacattttgatcactgacaagaagatctcaaacattcaagatatcttgccacttcttcaagaaattgttcaacaaggtaagtcattattgatcattgctgatgacgttactggtgaagctcttccaacacttgtattgaacaagattcgtggtaccttcaacgttgtagctgtt

SEQ ID NO: 43 - DNA, 552 bp, Unknown
  Uncultured human vaginal bacterium
gcaacgttacttgcacaggctttgattcgtgagggcatgaaaaacgttacggcaggtgcaaacccgatgatcctccgcaaaggtgtccagggtgctgtagatgtcgcggtaaaggcagttacggagcattccaaaaaagttagcggttccgaagatatcgcacgtgtcgcgacaatttcttctggagatgcaaccatcggcacattaattgcagaggcaatggaaaaagtgacgtcagatggtgtcatcacagtggaagaatccaaaactgcggagacatattccgaagtcgtcgaaggaatgatgtttgaccgcggatatatcacgccgtatatggttaccgatacggataagatggttgccgagattgatgacgcttatatcctgattaccgataaaaagatttccaacattcaggaaatccttccgcttctcgagaaaattgttcaggcgggtaagaaactcctgattattgcagaggatgtcgaaggtgaggcacttacgaccctgattctcaataaactgcgcggtacgtttacctgcgttgcggta

SEQ ID NO: 44 - DNA, 552 bp, Unknown; identical to SEQ ID NO: 24
  Uncultured human vaginal bacterium

SEQ ID NO: 45 - DNA, 553 bp, Unknown
  Uncultured human vaginal bacterium
gcaactgttttgactcaagcaattactaatgaaggtatgaagaacgtaactgctggtgctaacccagttggcattcgtcgtggtattgaaaaagctactgaagctgctgttaatgaattgcacaagattagccacaaggttgaatcaagagaccaaattgctaacgtagctgcagtttcatcatcttcaaaggaagttggtaacttaatcgctgacgctatggaaaaggttggtcacgatggcgttatcactattgaagattcacgtggtatcaacactgaactttcagtagttgaaggtatgcaatttgatcgtggttacttatcacaatacatggtaactgacaacgataagatggaagcagatcttgataatccttacattttgatcactgacaagaagatctcaaacattcaagatatcttgccacttcttcaagaaattgttcaacaaggtaagtcattattgatcattgctgatgacgttactggtgaagctcttccaacacttgtattgaacaagattcgtggtaccttcaacgttgtagctgttm

SEQ ID NO: 46 - DNA, 555 bp, Unknown
  Uncultured maggot midgut bacterium
gcaaccgtactggctcagtctatcatcactgaaggcctgaaagccgttgctgcgggcatgaacccgatggatctgaaacgcggtatcgataaagccgttgttgcagccgttgaagaactgaaaaaactgtctgcaccatgctctgataccactgcaatcgcgcaggtaggtactatctctgcaaactccgatgaaaccgtcggtaaactgattgcagaagcaatggataaagtcggcaaagaaggcgttatcactgttgaagaaggcaccggcctggaagatgaactggatgttgttgaaggtatgcagttcgaccgcggttacctctccccgtatttcatcaacaaaccggaaaccggttctgttgaattagaaaacccgtacatcctgctggtcgacaaaaaaatctccaacatccgtgaactgctgccggttctggaaggcgttgccaaagcaagcaaaccgctgatgatcatcgcagaagatgttgaaggtgaagcgctggcaacactggtggttaacaccatgcgcggtatcgtgaaagtggctgcagtg

SEQ ID NO: 47 - DNA, 555 bp, Unknown
  Uncultured maggot midgut bacterium
gcaactgttttggcacaagctatcgtgatcgaaggtttgaaagcagttgcagcgggtatgaacccaatggatctgaaacgaggtattgataaagcagttattgcagcagtagaagagctgaaaacactgtctgtcccatgttcagattctaaagcaattgctcaggttggtactatttctgcaaactctgacgaaaccgttggtaagctgatcgcagaagcaatggataaagtcggtaaagaaggcgttatcaccgttgaagaaggtactggtttagaagacgaactggatgttgttgaaggtatgcagttcgaccgtggttacttgtctccttatctcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 48 - DNA, 555 bp, Unknown
  Uncultured maggot midgut bacterium
gcaactgttttggcacaagctatcgtgatcgaaggtttgaaagcagttgcagcgggtatgaacccaatggatctgaaacgaggtattgataaagcagttattgcagcagtagaagagctgaaaacactgtctgtcccatgttcagattctaaagcaattgctcaggttggtactatttctgcaaactctgacgaaaccgtcggtaagctgatcgcagaagcaatggataaagtcggtaaagaaggcgttatcaccgttgaagaaggtactggtttagaagacgaactggatgttgttgaaggtatgcagttcgaccgtggttacttgtctccttatttcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 49 - DNA, 555 bp, Unknown
  Uncultured maggot midgut bacterium
gcaactgttttggcacaagctatcgtgatcgaaggtttgaaagcagttgcagcgggtatgaacccaatggatctgaaacgaggtattgataaagcagttattgcagcagtagaagagctgaaaacactgtctgtcccatgttcagattctaaagcaattgctcaggttggtactatttctgcaaactctgacgaaaccgttggtaagctgatcgcagaagcaatggataaagtcggtaaagaaggcgttatcaccgttgaagaaggtactggtttagaagacgaactggatgttgttgaaggtatgcagttcgaccgtggttacttgtctccttatttcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 50 - DNA, 555 bp, Unknown
  Uncultured maggot midgut bacterium
gcaaccgcactggctcagtctatcatcactgaaggcctgaaagccgttgctgcgggcatgaacccgatggatctgaaacgcggtatcgataaagccgttgttgcagccgttgaagaactgaaaaaactgtctgcaccatgctctgataccactgcaatcgcgcaggtaggtactatctctgcaaactccgatgaaaccgtcggtaaactgattgcagaagcaatggataaagtcggcaaagaaggcgttatcactgttgaagaaggcaccggcctggaagatgaactggatgttgttgaaggtatgcagttcgaccgcggttacctctccccgtatttcatcaacaaaccggaaaccggttctgttgaattagaaaacccgtacatcctgctggtcgacaaaaaaatctccaacatccgtgaactgctgccggttctggaaggcgttgccaaagcaagcaaaccgctgatgatcatcgcagaagatgttgaaggtgaagcgctggcaacactggtggttaacaccatgcgcggtatcgtgaaagtggctgctgtt

SEQ ID NO: 51 - DNA, 555 bp, Unknown
  Uncultured maggot midgut bacterium
gcaacagtattggcacaagcaattgtacgtgaaggtatgaaagcagtatctgctggtatgaacccaatggatatcaagcgtggtattgataaagcagttgcagcagcagttgcggagttgagagcaatttctaagccatgttctgatgataaatcaattgcacaagtaggtacgatttctgcgaactctgatgaagagattggtaaaatcatcgctgatgcaatggctaaagtaggtaaagaaggtgtaatcaccgttgaagaaggttctggtttagagaacgatttaacaacagttgatggtatgcaatttgatcgtggttacttgtctccttacttcatcaacaaccaacaatcaatggctgctgaattagacaatccattgatcttgatttgcgataagaaaatttctaacatccgcgaaatgttgacagttttagaagctgtagcaaaaacaggtcgcccattattgattgttgctgaagatgttgaaggcgaagcattggcaacattagtaatcaataacatgcgtggtatcgttgaagttgctgcggtt

SEQ ID NO: 52 - DNA, 555 bp, Unknown
  Uncultured maggot midgut bacterium
gcaaccgtactggctcaagctattgtgaatgaaggcctgaaagctgttgctgctggcatgaacccaatggatctgaagcgcggtattgataaagcagttatcgctgcagttgaagaactgaaagcgttatctgtaccttgctctgacaccaaagctatcgctcaggttggtactatctcagctaactccgatgagactgtaggtaaactgatcgcagaagcgatggacaaagtcggtaaagaaggcgttatcaccgttgaagacggtactggcctggaagatgagctggacgtagttgaaggtatgcagtttgaccgtggctacctgtctccttacttcatcaacaagcctgagactggcgctgtagaactcgaaagcccgttcatcctgctggctgacaaaaaaatctctaacatccgtgaaatgctgccagttctggaagcggttgctaaagccggtaaaccattgctgatcatcgctgaagatgttgaaggtgaagctctggcgactctggtagttaacaccatgcgcggtattgttaaagttgctgcggtt

SEQ ID NO: 53 - DNA, 555 bp, Unknown
  Uncultured maggot midgut bacterium
gcaaccgtactggctcagtctatcatcactgaaggcctgaaagccgttgctgcgggcatgaacccgatggatctgaaacgcggtatcgataaagccgttgttgcagcagtagaagagctgaaaacactgtctgtcccatgttcagattctaaagcaattgctcaggttggtactatttctgcaaactctggcgaaaccgttggtaagctgatcgcagaagcaatggataaagtcggtaaagaaggcgttatcaccgttgaagaaggtactggtttagaagacgaactggatgttgttgaaggtatgcagttcgaccgtggttacttgtctccttatttcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 54 - DNA, 555 bp, Unknown
  Uncultured maggot midgut bacterium
gcgactgtacttgcgcaagcaatcttaaacgaagggatcaaatctgtaaccgcaggtatgaacccaatggatttaaaacgcgggatcgatcttgcagttaaagcattggttgctgaaattaaagcaactgctaaacctgcgtcagataccaaagcgattgaacaagtgggttcgatttctgcaaactctgacgaaaccgttggtaagctgatcgcagaagcaatggataaagtcagtaaagaaggcgttatcaccgttgaagaaggtactggtttagaagacgaactggatgttgttgaaggtatgcagttcgaccgtggttacttgtctccttatttcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 55 - DNA, 555 bp, Unknown
  Uncultured maggot midgut bacterium
gcaacagtattggcacaagcaattgtacgtgaaggtatgaaagcagtatctgctggtatgaacccaatggatatcaagcgtggtattgataaagcagttgcagcagcagttgcggagttgagagcaatttctaagccatgttctgatgataaatcaattgcacaagtaggtacgatttctgcgaactctgatgaagagattggtaaaatcatcgctgatgcaatggctaaagtaggtaaagaaggtgtaatcaccgttgaagaaggttctggtttagagaacgatttaacaacagttgatggtatgcaatttgatcgtggttacttgtctccttacttcatcaacaaccaacaatcaatggctgctgaattagacaatccattgatcttgatttgcgataagaaaatttctaacatccgcgaaatgttgacagttttagaagctgtagcaaaaacaggtcgcccattattgattgttgctgaagatgttgaaggcgaagcattggcaacattagtaatcaataacatgcgtggtatcgttaaagttgctgcggtt

SEQ ID NO: 56 - DNA, 555 bp, Unknown
  Uncultured maggot midgut bacterium
gcaaccgcactggctcagtctatcatcactgaaggcctgaaagccgttgctgcgggcatgaacccgatggatctgaaacgcggtatcgataaagccgttgttgcagccgttgaagaactgaaaaaactgtctgcaccatgctctgataccactgcaatcgcgcaggtaggtactatctctgcaaactccgatgaaaccgtcggtaaactgattgcagaagcaatggataaagtcggcaaagaaggcgttatcactgttgaagaaggcaccggcctggaagatgaactggatgttgttgaaggtatgcagttcgaccgcggttacctctccccgtatttcatcaacaaaccggaaaccggttctgttgaattagaaaacccgtacatcctgctggtcgacaaaaaaatctccaacatccgtgaactgctgccggttctggaaggcgttgccaaagcaagcaaaccgctgatgatcatcgcagaagatgttgaaggtgaagcgctggcaacactggtggttaacaccatgcgcggtatcgtgaaagtggctgcagtg

SEQ ID NO: 57 - DNA, 555 bp, Unknown
  Uncultured maggot midgut bacterium
gcaactgttttggcacaagctatcgtgatcgaaggtttgaaagcagttgcagcgggtatgaacccgatggatctgaaacgaggtattgataaagcagttattgcagcagtagaagagctgaaaacactgtctgtcccatgttcagattctaaagcaattgctcaggttggtactatttctgcaaactctgacgaaaccgttggtaagctgatcgcagaagcaatggataaagtcggtaaagaaggcgttatcaccgttgaagaaggtactggtttagaagacgaactggatgttgttgaaggtatgcagttcgaccgtggttacttgtctccttatttcatcaacaaacatgaagcgggttcagttgaactggaaaatccattcgttctgttagttgataaaaaagtgtctaacatccgtgaattactgccagtattagaaggtgttgctaaagcaagcaagccacttcttatcatcgctgaagatgttgaaggtgaagcgctggcaactctggttgtgaacacaatgcgcggtatcgtgaaagtggctgctgtt

SEQ ID NO: 58 - DNA, 555 bp, Unknown
  Uncultured maggot midgut bacterium
gcaaccgtactggctcaagctattgtgaatgaaggcctgaaagctgttgctgctggcatgaacccaatggatctgaagcgcggtattgataaagcagttatcgctgcagttgaagaactgaaagcgttatctgtaccttgctctgacaccaaagctatcgctcaggttggtactatctcagctaactccgatgagactgtaggtaaactgatcgcagaagcgatggacaaagtcggtaaagaaggcgttatcaccgttgaagacggtactggcctggaagatgagctggacgtagttgaaggtatgcagtttgaccgtggctacctgtctccttacttcatcaacaagcctgagactggcgctgtagaactcgaaagcccgttcatcctgctggctgacaaaaaaatctctaacatccgtgaaatgctgccagttctggaagcggttgctaaagccggtaaaccactgctgatcatcgctgaagatgttgaaggtgaagctctggcgactctggtagttaacaccatgcgcggtattgttaaagttgctgcggtt

(19) United States
(12) Patent Application Publication
(10) Pub. No.: US 2005/0026188 A1
(43) Pub. Date: Feb. 3, 2005

(54) Methods of identifying, characterizing and comparing organism communities

(76) Inventors: Andrew G. Van Kessel, Saskatoon (CA); Sean Mathias Hemmingsen, Saskatoon (CA); Janet E. Hill, Saskatoon (CA)

Correspondence Address: TRASK BRITT, P.O. BOX 2550, SALT LAKE CITY, UT, 84110, US

(21) Appl. No.: 10/857,542

(22) Filed: May 28, 2004

Related U.S. Application Data
(60) Provisional application No. 60/474,471, filed on May 30, 2003.

(30) Foreign Application Priority Data
May 30, 2003 (CA) 2,430,586

(51) Int. Cl. C12Q 1/68; C12P 19/34

(52) U.S. Cl. 435/6; 435/91.2

(57) ABSTRACT

Methods of characterizing and identifying microorganisms within a community of microorganisms are described. The methods include amplifying variable regions of the cpn60 gene of samples taken from the microbial community. Primers designed from the analysis of phylogenetic comparisons of nucleotide sequences of the variable regions of the cpn60 gene are also disclosed.

### CROSS-REFERENCE TO RELATED APPLICATIONS

Pursuant to 35 U.S.C. §119(e), this application claims the benefit of U.S. Provisional Application 60/474,471, filed May 30, 2003, the contents of the entirety of which are incorporated by this reference.

### TECHNICAL FIELD

The invention relates generally to biotechnology, and more specifically to the characterization and identification of microorganisms within a community of microorganisms. The community of microorganisms may be isolated from any number of sources including, but not limited to, humans, animals, plants, soil, and other environments known to harbor microorganisms. The invention describes a method for identifying or characterizing groups of microorganisms within a sample of microorganisms within the community. The invention further discloses a method of comparing and analyzing the microorganisms using genetic sequences relating to specific variable regions flanked by non-variable, or constant, regions of nucleic acid within the cpn60 gene.

### BACKGROUND

Microorganism culture-based techniques were developed that, when combined with differentiation of isolates based on numerous physiological and biochemical tests, became the standard method for investigating a microbial community composition. A limitation of these methods is referred to as the great plate count anomaly (Staley, et al. 1985. Annu. Rev. Microbiol. 39:321-346). That is, only a small fraction of microorganisms present in a population can be cultured in the laboratory, wherein the fraction may be as low as 0.001 to 15%, depending on the community (Amann, et al. 1995. Microbiol. Rev. 59:143-169).

The development of recombinant DNA methods yielded a proliferation in small-scale studies of complex microbial communities, such as those associated with termite guts (Paster, et al. 1996. Appl. Environ. Microbiol. 62:347-352), rice paddy soil (Bai, et al. 2000. Microb. Ecol. 39:273-281), 120-million-year-old amber (Greenblatt, et al. 1999. Microb. Ecol. 38:58-68), Antarctic lake ice (Gordon, et al. 2000. Microb. Ecol. 39:197-202), and leaves of a seagrass in the northern Gulf of Eilat (Weidner, et al. 2000. Microb. Ecol. 39:22-31). Molecular methods for microbial community analysis (as reviewed in: Ranjard, et al. 2000. Res. Microbiol. 151:167-177; Theron, et al. 2000. Crit. Rev. Microbiol. 26:37-57; and Vaughan, et al. 2000. Curr. Issues Intest. Microbiol. 1:1-12) include denaturing gradient gel electrophoresis, temperature gradient gel electrophoresis, and restriction fragment length polymorphism analysis. While these methods disclose rapid comparative analyses of populations and generate population “fingerprints,” the methods do not identify individual organisms within the populations.

Methods that identify individual members of microbial communities are based on PCR and direct sequencing or cloning and sequencing of specific targets within microbial genomes. The most frequently used target is the 16S rRNA gene (Olsen, et al. 1986. Annu. Rev. Microbiol. 40:337-365, Pace, et al. 1986. Adv. Microb. Evol. 9:1-55). The molecular phylogenetic view of the microbial world is dominated by 16S rRNA sequence relationships, and the wealth of sequence information accumulated for 16S rRNA genes from thousands of organisms is stored in the Ribosomal Database Project (Maidak, et al. 1999. Nucleic Acids Res. 27:171-173). The Ribosomal Database Project is a standard tool for studying microbial communities. Libraries of total genomic DNA extracted from a community of interest can be screened for rRNA genes. Further, libraries of PCR amplified rRNA genes or gene segments can be generated and sequenced. While studies of multiple libraries of 16s rRNA have been attempted (Leser D.L., 2002, Applied Environmental Microbiology 68: 673-690), this approach involves significant challenges which prompted the suggestion that a DNA microarray approach was the future of such work.

Other gene targets used in microbial identification and elucidation of phylogenetic relationships include rpoB (Dahllof, et al. 2000. Appl. Environ. Microbiol. 66:3376-3380),gyrB (Kasai, et al. 1998. Genome Inform. Ser. Workshop Genome Inform. 9:13-21), pmoA (Bourne, et al. 2001. Appl. Environ. Microbiol. 67:3802-3809), and cpn60 which encodes the 60-kDa chaperonin found in virtually all eubacteria and the mitochondria and chloroplasts of eukaryotes (Sigler, et al. 1998. Annu. Rev. Biochem. 67:581-608).

The overriding limitation to sequence-based studies of a community of organisms is scale. The most thorough, direct analysis of cloned 16S rRNA gene sequences from a complex microbial community involved the sequencing of 284 16S rRNA gene fragments from a human fecal sample (Suau, et al. 1999. Appl. Environ. Microbiol. 65:4799-4807). A study of this fecal sample resulted in the identification of 82 different 16S rRNA sequences and is not adequate to catalog the microbial diversity in feces thought to contain at least 500 different bacterial species (Moore, et al. 1974. Appl. Microbiol. 27:961-979), or soil, estimated to contain approximately 13,000 different species (Torsvik, et al. 1990. Appl. Environ. Microbiol. 56:782-787).

The development of high-throughput technologies for genomics applications presents an opportunity to conduct large-scale, even comprehensive, studies of complex microbial communities. It is now possible to conduct a study of the application of genomics technology and the cpn60 molecular diagnostic method to cataloging the diversity in a microbial community.

### SUMMARY OF THE INVENTION

The invention includes a method for profiling the relative and absolute abundance of different kinds of organisms in a population. The method involves, generally, the identification of a target nucleic acid region that is present in most organisms of interest, wherein the nucleic acid sequence of the nucleic acid region varies between different organisms of interest.

In one embodiment, the population as a whole is sampled and the target nucleic acid region is extracted, without the need to separate different kinds of organisms before the nucleic acid extraction. The extracted nucleic acid is examined to identify which target regions it contains. The target regions are compared to known target region sequences for organisms of interest to identify what organisms of interest are present in the population and what their relative abundance is. In many cases, once the target regions are identified, the organisms may be grouped phylogenetically so that the relative abundance of different general types or classes of organisms may be compared. Once a population has been profiled, this information may be used to identify potential concerns relating to the population or to predict further population shifts.

The development of high-throughput technologies for genomics applications presents an opportunity to conduct large-scale, even comprehensive, studies of complex communities of organisms. The communities may comprise intestinal flora, vaginal flora, midgut flora, biofilm flora, soil flora, Gram-positive flora, Gram-negative flora, mammalian flora, animal flora, animal organ flora, feces flora, wastewater treatment flora, brewing flora, water flora, industrial flora, cooling water flora, sewage processing pond flora, plant surface flora, flora involved in industrial processes, food flora, flora associated with food, and combinations of any thereof. It is also possible to conduct a study of the application of genomics technology and the cpn60 molecular diagnostic method to cataloging the diversity in a microbial community. Results (below) confirm the potential for this method in larger studies of microbial communities and the establishment of cpn60 as a universal target for studying the phylogenetic relationships of microorganisms in complex communities.

One potentially useful application of these new methodologies concerns the use of antibiotics in mammalian feed stocks. Modern North American pig rearing practices involve the inclusion of subclinical doses of antibiotics in feed to control populations of pathogenic bacteria. Pigs undergoing weaning and the coincident shift from a predominantly Gram-negative to a predominantly Gram-positive flora are particularly vulnerable to pathogens such as Escherichia coli and Clostridium perfringens. With increasing pressures to eliminate the use of antibiotics to control intestinal diseases in pigs comes an increasing interest in understanding the role of normal intestinal flora in mammalian health and in utilizing the potential prebiotic properties of feed ingredients.

Genomics-inspired technologies such as robotic colony picking, template preparation, sequencing, and automated data assembly and analysis maybe employed to produce potentially comprehensive profiles of important microbial communities. The libraries of sequence data produced will be tools for developing methods to quantitate organisms within a population, for the detection of pathogens or specific organisms of interest, to monitor changes in populations over time or treatment, and for creating specific probes for techniques, such as fluorescence in situ hybridization. In one embodiment, the probe may be labeled with a detectable marker such that the probe may be used to detect sequences complementary to the probe. Non-limiting examples of technologies used to create detectable markers include labeling the probe with a radioactive substance, a marker capable of detection with fluorescence, a marker capable of binding an antibody, and other techniques of detecting a labeled probe.

The potential extension of these new technologies to identifying unique microorganisms in human, animal and plant subjects provides an opportunity to develop novel diagnostic kits. Rapid and accurate identification of microorganisms within mammals allows rapid and more effective treatment of mammalian subjects. The savings in time potentially yields new life saving measures through more efficient targeting of microorganisms using selective anti-microbial agents.

Concerns may arise when the ratio of different types of organisms in the population deviate from what is observed in normal, healthy subjects. Thus, the invention allows the comparison of population profiles to be obtained from subjects who may be at risk to those from healthy subject and (if desired) subjects having known population profile abnormalities of clinical or other concern.

The target nucleic acid region may be any region present within most organisms of interest and capable of ready identification from a heterogeneous sample. In some instances, the target nucleic acid variable region is flanked by non-variable regions which allow PCR amplification of the variable region and the production of libraries. Target regions of particular interest may be found in cpn60 (chaperonin 60), 16s rRNA, 23s rRNA and the 16s-23s interspacer region.

In one embodiment, a complete inventory or census of the population of interest is developed, wherein the inventory or census is based on partial cpn60 sequences and is as complete as possible. To do this, the total genomic nucleic acid from the population is used as a template in PCR reactions and the universal cpn60 PCR primers (SEQ ID NOS: 1 and 2) H279 and H280 (originally described in U.S. Pat. Nos. 5,708,160 and 5,989,821 to Goh et al.) are used as the primers. These primers amplify 552, 555 or 558 nucleotides (some other very rare exceptions to these sizes) of the cpn60 gene from the genomes present. The PCR product pool is ligated into a cloning vector to create the library or libraries, and randomly selected clones are sequenced. The result of the sequencing is a collection of partial, “universal target” sequences representing population constituents. Putative taxonomic assignments of the sequences are made based on comparing each sequence to the reference database of cpn60 sequences. The frequency with which each sequence is recovered from the library is determined. This sequence collection of cpn60 universal targets becomes the jumping-off point for all downstream activities.

In another embodiment, representatives of each unique sequence are used to create a phylogenetic tree and the frequencies of each sequence from each library are applied to the tree. Clusters or branches of the tree (clusters or groups of phylogenetically related sequences) may be identified which have a ratio of interest. Alternatively, a taxon of interest that is many times more abundant in one of the libraries may be identified. Clusters or branches of the tree may also be designated as interesting if they are present in constant amounts in all libraries. Once a target taxon has been identified as interesting, this subset of sequences may be pulled from the collection for further analysis.

In a further embodiment, signature sequences in the target group that are common to all members of that group may be found, wherein the signature sequences are not found in other population members (and preferably not in anything unrelated in the known universe of cpn60 sequences). These signature sequence elements may be used to design PCR primers, or oligonucleotides, which amplify a region internal to the universal target (552-558 bp). The size of the PCR product will be less than 552-558 bp, but the particular size will depend on the possible location of distinguishing primers. In some instances a product size of 100-200 bases may be generated which is suitable for quantitative PCR such as, for example, Taqman type real-time PCR. If SYBR green or another approach is used for quantitative PCR, the product size will be less important. The PCR products produced by the universal primers could also be analyzed using techniques such as denaturing gradient gel electrophoresis (DGGE), as has been often done in 16s rRNA based studies, in addition to or in place of the sequencing.

In another embodiment, the present invention discloses methods for identifying specific groups of microorganisms within a diverse community based on the heterogeneity of specific regions within the cpn60 gene and the ubiquitous presence of the cpn60 gene in various organisms. The use of the cpn60 gene of the present invention has advantages over known techniques, e.g., use of the 16S rRNA genetic marker, in that for closely related organisms, more phylogenetic information exists in the protein-encoding cnp60 gene sequence in relation to the structural RNA-encoding 16S rRNA gene. Thus, the invention further includes the design and use of primers, or oligonucleotides, for the identification of organisms within, and the characterization of, a microbial community using the cpn60 gene.

The amplification and sequencing of cpn60 amplificates having a cpn60 variable region from a microbial community, or a population within the community, allows primers characteristic of the cpn60 homologues within the microbial population to be designed. Thus, in another embodiment, described herein are methods for designing population- or taxa-specific primers. The primers may be useful in determining the number, types, and identities of specific populations of microorganisms present within the community or environment. The disclosed methods for analyzing microbial populations allows for rapid determination of causative agents such as, for example, in disease, malnutrition, and infection. Kits including these primers may also be designed and implemented in the rapid identification of certain microorganisms.

A set of primers may be designed using sequences that may be designated as “signature sequences.” The primers will typically be about 15-25 nucleotides in length and a set of primers will have similar predicted melting temperatures. The designed primers will amplify sequences of the organisms within the “target group” using a set of amplification conditions and will fail to amplify sequences of organisms outside of the “target group” under similar amplification conditions.

In one embodiment, a method for characterizing a community of organisms is disclosed. The method comprises pooling nucleic acid from the community of organisms. Portions of the pooled nucleic acid corresponding to a conserved gene are amplified using primers specific for the conserved gene to produce a set of amplificates. In one embodiment, the amplified conserved gene is cpn60 (homologue of hsp60 and E. coli groEL genes) and the primers used to amplify the gene comprise: 5′-GAI III GCI GGI GA(C/T) GGI ACI ACI AC-3′ (SEQ ID NO: 1) 5′-(C/T)(G/T)I (C/T)(G/T)ITCI CC(A/G) AAI CCI GGI GC(C/T) TT-3′ (SEQ ID NO: 2) as disclosed in U.S. Pat. Nos. 5,989,821 and 5,708,160, which are incorporated herein by this reference in their entirety. The amplificates are cloned by ligating the amplificates into a vector and transforming the vector into a cell, wherein the replicated vectors may be harvested for subsequent use. The amplificates, either by themselves or from the replicated vectors, may be sequenced. The obtained sequences are compared to known sequences by phylogenetic comparison algorithms yielding genetic relationships that may be used to characterize the organisms from within the community. Organisms within the community may be grouped and identified based on different relationships.

The methods for characterizing a community of organisms may be used to study a variety of different stimuli on the community. For instance, a community of microorganisms may be characterized as a “normal,” or control, community of microorganisms. The community may comprise an organ of an animal, an animal, a sample of water or soil, a biofilm, or any other environment of organisms. A stimuli may be added to the community or a community may be exposed to an exogenous variable, such as the “test” community, wherein the test community may be characterized using the methods for characterizing a community of organisms described herein. The characterization of the “test” community may be compared to the characterization of the “normal” community and the difference may be studied. The test community may be from a malnourished, diseased, or pathogen infected organism, wherein the “normal” community is not malnourished, diseased, or infected and is otherwise healthy.

Also disclosed is, a method for generating a set of primers specific for an organism or a group of organisms in a community. The method includes pooling nucleic acid from the community of organisms. Portions of the pooled nucleic acid corresponding to a conserved gene, such as cpn60, are amplified using primers specific for the conserved gene to produce a set of amplificates. The amplificates are cloned by ligating the amplificates into a vector and transforming the vector into a cell, wherein the replicated vectors are harvested for subsequent use. The amplificates, either by themselves or from the replicated vectors, are sequenced. The obtained sequences are compared to known sequences such that phylogenetic relationships and comparisons are made in order to characterize the organisms from within the community. Organisms within the community are grouped and identified based on different relationships. Target nucleotide sequences of a “target group” of organisms common to the organisms within the “target group” and different from organisms outside of the “target group,” that is, the nucleotide sequences that will selectively amplify only those target nucleotide sequences within the target group and not those outside the target group are designed.

The target nucleotide sequences, or target primers, can be used to design primers for the target group. The primers may be used to characterize the target group within the community. For instance, quantitative PCR using the target primers may be performed to enumerate the microorganisms present in the target group or the abundance or the target group within the community.

In one exemplary embodiment, a method of designing population- or taxa-specific primers includes isolating a nucleic acid sample from a host organism, developing a library from the isolated samples having nucleotide sequences corresponding to a variable region within the cpn60 gene that is flanked by constant regions that are conserved among cpn60 homologues. The method further includes developing a list of nucleotide sequences in the library of cpn60 genes and comparing the nucleotide sequences to the list of cpn60 genes. A signature sequence characteristic of the nucleotide sequences found in the library may also be determined wherein group-, or taxa-, specific primers that correspond to the signature sequence may be designed based on the signature sequence. The group primers may be used to amplify, detect or quantitatively amplify, i.e., quantitative PCR, the variable regions of the nucleic acid of members of the group, wherein the variable regions of the nucleic acid of organisms outside of the specific group are not amplified.

The present invention includes a kit for the purpose of identifying unique strains of microorganisms within a diverse and large community of microorganisms. Such a kit preferably comprises a carrier means being compartmentalized to receive in close confinement one or more container means such as vials, tubes, and the like, each of the container means comprising one of the separate elements to be used in the method. For example, one of the container means may comprise means for amplifying target DNA including the necessary enzyme(s) and oligonucleotide primers for amplifying the target DNA from the sample obtained from the host. The oligonucleotide primers include primers having a sequence similar or identical to SEQ ID NO: 3 through SEQ ID NO: 5 (see Table 5) and SEQ ID NO: 7 through SEQ ID NO: 9 (see Table 5) or primer sequences substantially complimentary thereto.

Methods of determining a ratio corresponding to the relative abundance of a group of taxonomically related microorganisms within a community of microorganisms is also disclosed. The method includes isolating a nucleic acid sample from an environment and developing a library of nucleotide sequences from the nucleic acid sample that includes nucleotide sequences corresponding to variable regions of the cpn60 gene. A listing of nucleotide sequences of the library of cpn60 genes is generated and a comparison of the nucleotide sequences in the list of cpn60 genes is made to a known cpn60 database. A signature sequence characteristic of the nucleotide sequences found in the developed library is generated such that group- or taxa-specific primers corresponding to the signature sequence may be designed. The group specific primers may be used to amplify variable regions of nucleic acid of members of the community of microorganisms, wherein the group specific primers will not be able to amplify variable regions of microorganisms outside of the group. The group specific primers may also be used to amplify the cpn60 nucleotide variable regions from the sample in a controlled manner in order to quantitate the relative numbers of microorganisms in the community or in the group.

Results (below) confirm the potential for this method in larger studies of microbial communities and the establishment of cpn60 as a universal target for studying the phylogenetic relationships of microorganisms in complex communities.

One potentially useful application of these new methodologies concerns the use of antibiotics in mammalian feed stocks. Modern North American pig rearing practices involve the inclusion of subclinical doses of antibiotics in feed to control populations of pathogenic bacteria. Pigs undergoing weaning and the coincident shift from a predominantly Gram-negative to a predominantly Gram-positive flora are particularly vulnerable to pathogens such as Escherichia coli and Clostridium perfringens. With increasing pressures to eliminate the use of antibiotics to control intestinal diseases in pigs comes an increasing interest in understanding the role of normal intestinal flora in mammalian health and in utilizing the potential prebiotic properties of feed ingredients.

Genomics-inspired technologies such as robotic colony picking, template preparation, sequencing, and automated data assembly and analysis maybe employed to produce potentially comprehensive profiles of important microbial communities. The libraries of sequence data produced will be tools for developing methods to quantitate organisms within a population, for the detection of pathogens or specific organisms of interest, to monitor changes in populations over time or treatment, and for creating specific probes, or oligonucleotides, for techniques, such as fluorescence in situ hybridization.

The potential extension of these new technologies to identifying unique microorganisms in human subjects provides an opportunity to develop novel diagnostic kits. Rapid and accurate identification of microorganisms within mammals allows rapid and more effective treatment of mammalian subjects. The savings in time potentially yields new life saving and humane measures through more efficient targeting of microorganisms using selective anti-microbial agents.

### BRIEF DESCRIPTION OF THE DRAWINGS

This patent or application file contains at least one drawing executed in color. Copies of the patent or patent application publication with color drawing(s) will be provided by the Office upon request and payment of the necessary fee.

FIG. 1A. Frequency distribution of unique nucleotide sequences recovered from the combined pig feces cpn60 libraries. FIG. 1B. Taxonomic breakdown of total library contents. Assignment to a taxonomic group was based on comparisons of clone sequences to a database of cpn60 reference sequences.

FIG. 2. Phylogenetic relationships of 280 unique Cpn60 peptide sequences translated from 398 unique nucleotide sequences. Distance calculations were made using the Dayhoff PAM matrix and the dendrogram was produced by neighbor-joining. The scalebar represents 0.1 substitutions per site. Branches are colored according to the assigned taxonomic group of the sequences (red, CFB group; green, Proteobacteria gamma; blue, bacillus/clostridium group; black, other).

FIG. 3. Phylogenetic relationships of 12 clone peptide sequences assigned to the CFB group, including the two most abundant cloned sequences (represented by 001_f12 and 002_a03). The tree is a consensus of 100 neighbor-joined trees. Distance calculations were made using the Dayhoff PAM matrix and branch lengths were imposed on the consensus tree using fitch. Nodes with bootstrap values >50% are indicated with white dots. Reference sequences used in the tree are Flavobacterium hydatis (GenBank accession AAK32145), Flavobacterium ferrugineum (AAK32146), Bergeyella zoohelcum (ATCC43767), Chryseobacterium meningosepticum (ATCC13253), Chryseobacterium gleum (ATCC35910), Bacteroides forsythus (CAB43992), Bacteroides vulgatus (ATCC8482), Bacteroides uniformis (ATCC8492), Bacteroides ovatus (ATCC8483), Prevotella bivia (ATCC29303), Prevotella intermedia (ATCC25611), Rhodothermus marinus (strain ITI 376, AAD37976), and Chlorobium tepidum (derived from contig 3499, TIGR unfinished genome database).

FIG. 4. Cumulative frequency distribution plots for the pig feces library (solid line), a population of individual species from 172 different eubacterial and eukaryotic genera, a single taxonomic subclass (77 species from 34 genera of Proteobacteria gamma) and a single genus, Lactobacillus. Plots were generated from DNA identity matrices derived from CLUSTALw multiple sequence alignments using GeneDoc.

FIG. 5A. Frequency distributions of unique nucleotide sequences recovered from clone libraries P56, P40, B40 and B56. FIG. 5B. Taxonomic composition of libraries B40, B56, P40 and P56.

FIG. 6. Taxonomic composition of groups of library clones pooled by PCR annealing temperature used in library construction (left panel) or genomic DNA template extraction method (right panel).

FIG. 7 depicts in bar graph format the species population found in C, B and W libraries and depicts in bar graph format the number of clones recovered from the C, B and W libraries.

FIG. 8 illustrates the phylogenetic relationship between 136 sequences originating from the C library using a phylogenetic tree generated by phylogenetic analysis using a maximum likelihood distance calculation followed by the neighbor-joining method.

FIG. 9 illustrates the phylogenetic relationship of 171 unique nucleotide sequences derived from the B library using a phylogenetic tree generated by phylogenetic analysis using a maximum likelihood distance calculation followed by the neighbor-joining method.

FIG. 10 illustrates the phylogenetic relationship between 110 sequences originating from the W library using a phylogenetic tree generated by phylogenetic analysis using a maximum likelihood distance calculation followed by the neighbor-joining method.

FIG. 11 illustrates the phylogenetic relationship between 316 sequences derived from the C, B and W libraries using a phylogenetic tree generated by phylogenetic analysis using a maximum likelihood distance calculation followed by the neighbor-joining method. The ratios of clone frequency corresponding to each sequence group are also indicated (C:B:W).

FIG. 12 depicts a comparison of frequencies of recovery of clones corresponding to various taxonomic groups (genera) from human vaginal flora libraries BV1 and BV2 depicted in bar graph format.

FIG. 13 illustrates the phylogenetic analysis of Chlorobium-Flexibacter-Bacteriodes (CFB) group sequences recovered from human vaginal flora libraries (note: BV1-010 is identical in sequence to bv1-087 (SEQ ID NO: 29) and BV1-020 is identical in sequence to bv1-050 (SEQ ID NO: 20).

FIG. 14 depicts an experimental scheme for the creation of cpn60 sequence libraries from the midgut microflora of Delia radicum.

FIG. 15 illustrates the peptide sequence distance tree for sequences derived from Delia radicum midgut flora cpn60 library and selected cpn60 reference sequences.

### BEST MODE OF THE INVENTION

The present invention discloses methods for identifying a microorganism, or characterizing populations or groups of microorganism in a community through amplification of variable regions of a polynucleotide sequence encoding a chaperonin-60 protein (such as Cpn60, GroEL, or Hsp60) using PCR-based or related molecular approaches. The amplified nucleotide sequences MAY be utilized to identify and distinguish microorganisms at the species level, at a group level, or at a taxa-specific level.

The present invention discloses oligonucleotide primers for identifying microorganisms or groups of microorganisms within a large community of microorganisms by amplifying a polynucleotide sequence from a sample obtained from a community, wherein the amplified sequence encodes a variable region of a chaperonin-60 protein (such as Cpn60, GroEL, or Hsp60). Examples of polynucleotide sequences of interest that may be amplified include, but are not limited to, cpn60, 16s rRNA, 23s rRNA and the 16s-23s interspacer region. This is accomplished by using primers having a sequence that is substantially homologous to sequences of non-variable regions flanking the variable regions of a target gene, such as cpn60.

Oligonucleotide primers described herein may be isolated (pure), may be double or single stranded, may consist of DNA, RNA, or any other nucleic acid molecule substantially homologous to sequences flanking the cpn60 gene such that the primers may hybridize to the target region under stringent conditions and be used in the methods described herein. The length of the primers of the present invention may depend on many factors including, but not limited to, the identity of a host organism or environment, an amount of material available, techniques used, and other unforeseen variables. The primers should be able to hybridize to the locus to be amplified under conditions that allows accurate synthesis of nucleic acid, thus requiring the primers to be substantially complimentary to each strand of the non-variable regions flanking the variable regions within the genomic locus to be amplified.

As used herein, the term “organism” will be used to refer to any organism whose genome encodes the chaperonin-60 protein or its homologue, including microorganisms such as bacteria, fingi, protozoans, and other known microorganisms. Nucleic acid from the organism is prepared using any number of techniques known to isolate nucleic acid. The non-variable regions flanking the variable regions within the genomic locus to be amplified are annealed to the primers under conditions which allow efficient and accurate hybridization. Various techniques may be used to encourage maximum diversity in the selection of targets during the annealing process, including, but not limited to, splitting the target nucleic acid into equivalent fractions to be processed in parallel, wherein each fraction is processed at a different annealing temperature.

Target nucleic acid is amplified using any number of known nucleic acid amplification techniques, such as the addition of agents that catalyze reproduction of DNA such as E. coli DNA polymerase I, T4 DNA polymerase, Taq and Vent polymerases, reverse transcriptase, and other known enzymes. Products may be purified using a variety of known isolation methods. These products may be sequenced to identify the organisms from which the original variable sequence originated. The sequencing may be accomplished a number of ways, including, but not limited to, ligating the amplified variable regions containing flanking ends into a vector for the purpose of transfection into reproducing bacterial hosts (such as E. coli). These hosts may be plated and colonies isolated for the purpose of growing large quantities of the variable region carried by the plasmid within the host cells. The plasmids may be isolated from the bacterial host using any known plasmid isolation procedure, such that sequencing of the variable region and comparison of these sequences may be performed.

The aforementioned techniques are examples by which one of ordinary skill in the art is able to identify and compare sequences of members of a population within a community of organisms. One of ordinary skill in the art may be able to design other methods for isolating and amplifying the isolated variable regions for sequencing that would be equivalent as used in the methods described herein.

The methods of the present invention may be applied to a range of nucleic acid regions so long as (a) the nucleic acid region is present in the organisms of interest; (b) the nucleic acid region has non-variable regions flanking a variable region so as to allow selective amplification of the variable region; and (c) the nucleic acid region is phylogenetically informative (allowing association of the nucleic acid region with a particular species, genus or variety of organism.) For example, the DNA sequence encoding 16S rRNA is known to be present in a wide range of species, as are “universal primers” used to amplify a variable region in 16S rRNA suitable to allow phylogenetic identification of the source organism.

In some instances, the variable region may be between about 25-1,000 bases. In other instances, the variable region may be about 50-500 bases, and in others the variable region may be about 50-200 bases.

The invention described herein further discloses methods for designing primers specific for groups of organisms or target taxa found within communities of microorganisms. Upon comparison of the sequences to a database of cpn60 sequences (i.e., cpnDB), the neighbors of the identified and sequenced clones may be determined. The neighbors may be placed in a group for the purpose of designing a set of target sequences, or primers, specific for the target group. These target sequences may be used as starting materials for detecting the presence or absence of the unique group within various samples obtained from the community or environment.

Non-limiting examples of cpn60 sequences may be found with the following accession numbers: AF338228, AF274871, AF406639, AY263150, AY263147, and AY123725. In some instances, the target sequence may be a chaperonin sequence, such as a type 1 chaperonin sequence.

As used herein, a “cnp60-like” sequence is an oligonucleotide sequence having at least 60% homology to (a) the E. coli groEL sequence in prokaryotes or (b) the Candida albicans hsp60 sequence in eukaryotes, and is capable of encoding a protein which, when expressed under suitable conditions, has a biological function similar to that of cpn60, groEL or hsp60. In some instances, homology of at least 70%, 80% or 90% may be used.

As used herein, the term “cnp60 variant” refers to a cpn60-like sequence which is at least 90% homologous to the most highly expressed cpn60, groEL or hsp60 gene found in the organism being examined. As used herein the term “DNA region” refers to a continuous deoxyoligonucleotide sequence found within a genome of an organism (including nuclear, mitochondrial, chloroplast and plasmid DNAs). While the term DNA region may refer to a gene or a part of a gene, it may also refer to regulatory or structural DNA region, intron DNA, or unexpressed genes or pseudo genes.

As used herein, when a DNA region is said to be “represented in a species,” this means that the species has an oligonucleotide sequence having a high degree of homology with the DNA region and the oligonucleotide sequence performs substantially the same role as the DNA region. In some instances, the oligonucleotide sequence will be at least about 75% homologous to the DNA region. In some cases the homology will be at least about 80%, 85%, 90% or 95%. Complete sequence identity is not required.

The invention is further explained by the following examples. The following examples are intended as illustrative and are not intended to represent a limitation of the present invention. There exist numerous additional applications known by those or ordinary skill in the art. Additionally, the techniques described herein are intended as representations of examples of techniques which may be used to achieve the results enumerated herein. One of ordinary skill in the art would see that alternative and equally successful techniques may be used to achieve the same goals of each step of the procedure discussed above.

It will be apparent by those of ordinary skill in the art that the methods, primers and libraries of the present invention may be used in screening for, predicting, diagnosing or developing strategies for treating a wide range of human and animal conditions. Of particular interest are conditions associated with abnormal “flora” (abnormal populations of organisms). The populations may be abnormal due to the presence, absence or abnormal relative abundance of one or more species of organism.

Some examples of disorders associated with “abnormal” flora include without limitation: vaginosis or Inflammatory Bowel Diseases in humans (i.e., Chrohns's, colitis), necrotic enteritis in chickens (linked to Clostridium overgrowth, such as C. perfringens). Intestinal flora profiles are also related to livestock performance since the flora affects nutrient absorption and digestion. The microbial flora on the surface of plant tissue is also contemplated since it is thought to play a role in disease resistance and plant health.

Applications of the present invention outside the study of flora of human, animal or plant are also contemplated. For instance, it is frequently useful to know the profile of organisms in other environments. Examples of other environments that may be studies include without limitation: quantities of nitrogen-fixing microorganisms in agricultural soils, toxin-degrading microorganisms in the soil at sites of contamination (bioremediation) or biofilms. Further, industrial processes that depend on certain flora, such as pulp mill in-mill streams contain microorganisms vital to proper flocculation, may be studied using the present invention.

### EXAMPLES

### Example 1A

Pig feces are a tractable microbial community, rich in microbial life (estimated to exceed 10¹¹ organisms per g of feces, Finegold, et al. 1974. Am. J Clin. Nutr. 27:1456-1469, and Moore, et al. 1974. Appl. Microbiol. 27:961-979), for which there is a wealth of descriptive literature. The invention described herein may be applied to any number of various sources of microorganism communities as would be apparent by one of ordinary skill in the art.

Wheat and barley are the primary feed ingredients for some pigs, while corn is the major ingredient of feed in parts of North America. Wheat and barley have higher levels of non-starch polysaccharides than corn and could have an effect on the composition of gut microflora in mammals eating these diets. Total genomic DNA from the ideal contents samples from pigs fed antibiotic-free diets containing corn, wheat or barley as the primary energy source may be used to create cpn60 sequence libraries using the methods disclosed herein in order to investigate the effects of corn, wheat or barley-based diets on pig intestinal microflora. A phylogenetic analysis of the resulting sequence data may be used as the basis for designing primer-probe, i.e., oligonucleotides, sets to target specific taxonomic groups within the populations for quantitative PCR. A real-time quantitative PCR approach may be taken to validate the representation of the targeted sequences in the cloned libraries and to develop molecular tools for the monitoring of taxonomic groups of interest within the pig ileum populations. In an independent study using the same samples of digestive contents (digesta samples), bacterial populations may be enumerated using selective agar.

Pigs and feces collection.

Fecal samples were obtained from the recta of 6-week-old pigs (n=5) housed in a commercial swine facility (Prairie Swine Centre Inc., Saskatoon, Saskatchewan, Canada). A medicated (chlortetracycline at 308 mg/kg, sulfamethazine at 308 mg/kg, and penicillin at 154 mg/kg) wheat and soybean meal-based diet formulated to meet nutrient requirements was fed from 21 days of age (weaning). Fecal samples were pooled (a total of approximately 2 g) and stored at −20° C. until genomic DNA was extracted.

Genomic DNA extraction.

Two methods of genomic extraction were used. One method is a modification of the benzylchloride extraction method (Zhu et al. 1993. Nucleic Acids Res. 21:5279-5280), approximately 0.8 g of feces was thawed and dispersed in 5 ml of benzylchloride extraction buffer (100 mM Tris-HCl, pH 9.0, 40 mM EDTA). To 500 μl of the suspension was added 100 μl of 10% sodium dodecyl sulfate (SDS) and 300 μl of benzyl chloride. The remaining 4.5 ml of fecal suspension was reserved at −20° C. The sample was mixed by vortexing and incubated at 50° C. for 30 min, with vortexing at 5-min intervals. 300 μl of 3 M sodium acetate (pH 5) was added and the sample was mixed by inversion and incubated on ice for 15 min, followed by centrifugation at maximum speed in a microcentrifuge at 4° C. for 15 min to separate the aqueous and organic phases. The supernatant was transferred to a clean tube and nucleic acids were precipitated by the addition of 400 μl of isopropanol followed by centrifugation at top speed in a microcentrifuge for 10 min at 4° C. The pellet was washed in cold 70% ethanol, dried, and resuspended in 100 μl of TE (100 mM Tris-HCl, pH 8, 1 mM EDTA).

In another method, approximately 0.8 g of feces was dispersed in 5 ml of 25% sucrose-40 mM Tris, pH 8. To 500 μl of the suspension, 100 μl of lysozyme (10 mg/ml in 25 mM Tris, pH 8) was added, and the sample was incubated at 4° C. for 10 min, followed by the addition of 100 μl EDTA (0.5 M, pH 8) and incubation at 4° C. for 10 min. 1 ml of lysis buffer (62.5 mM EDTA, 50 mM Tris, pH 8, 1% (vol/vol) Triton X-100) was added and the sample was incubated at 4° C. for 15 min with periodic mixing. The lysate was extracted twice with 25:24:1 (vol/vol/vol) phenol-chloroform-isoamyl alcohol and nucleic acids were precipitated by the addition of 85 μl of 3 M sodium acetate and 850 μl of isopropanol, followed by centrifugation at maximum speed in a microcentrifuge for 10 min at 4° C. Pellet was washed once with 70% ethanol, air dried, and resuspended in 100 μl of TE.

PCR and cloning of PCR products.

Genomic DNA extracted from feces (1 μl of either benzylchloride or phenol-chloroform-extracted DNA) was used as the template in PCR reactions (PCRs). The PCR primers used were H279, 5′-GAI III GCI GGI GA(C/T) GGI ACI ACI AC-3′ (SEQ ID NO: 1), and H280, 5′-(C/T)(G/T)I (C/T)(G/T)I TCI CC(A/G) AAI CCI GGI GC(C/T) TT-3′ (SEQ ID NO: 2) (U.S. Pat. No. 5,989,821 incorporated into this application by reference). Inosine (I) was used to reduce the degeneracy of the sequences (Ohtsuka, et al. 1985. J. Biol. Chem. 260:2605-2608). Primers were designed to amplify the region between codons 92 and 277 based on the Escherichia coli groEL sequence (accession number X07850, groEL is the E. coli homologue of cpn60). Other primers that may be used have the following nucleotide sequences: 5′-GTTGTCGTACC(G/A)TCACCAGCAATTTC-3′ (SEQ ID NO: 10) and 5′-AA(G/A)GCGCCTGGTTT(C/T)GGTGAT(C/A)(G/A)(A/T/C/G)(C/A)(G/A)-3′ (SEQ ID NO: 11) (as recorded in U.S. patent application No. 5,7008,160, incorporated into this application by reference). The PCR reactions contained 50 mM KCl, 10 mM Tris-HCl (pH 8.3), 1.5 mM MgCl₂, 250 μM each of the four deoxynucleoside triphosphates, 2 U of Taq DNA polymerase, and 0.5 μg (50 pmol) of each primer.

PCRs were performed on a Stratagene Robocycler thermocycler according to the following parameters: 3 min at 95° C., 40 cycles of 1 min at 95° C., 1 min at 40° C., 1 min at 72° C., and 10 min at 72° C. PCRs included a negative control reaction containing no template DNA to ensure that no contaminating template was present in the reactions. An additional set of PCRs were done as described except that the annealing temperature was 56° C. The resulting four PCR products were agarose gel purified and ligated into vector pCR2.1-TOPO with the TOPO T-A cloning kit (Invitrogen), and Escherichia coli transformed with the vectors was plated on Luria-Bertani agar (LB) containing ampicillin and 5-bromo-4-chloro-3-indolyl-β-D-galactopyranoside (X-Gal). The resulting libraries were named according to the template extraction method and PCR annealing temperature used in their production: B56 and B40 (benzylchloride template amplified with an annealing temperature of 56° C. or 40° C., respectively); P56 and P40 (phenol-chloroform template amplified with an annealing temperature of 56° C. or 40° C., respectively). Colonies (576 white colonies from each library) were picked and used to inoculate 96-well plates containing 100 μl of LB with ampicillin (50 μg/ml) per well. Culture plates were incubated overnight in humidified containers at 37° C. with shaking. Glycerol (100 μl of 30% glycerol in LB) was added to each well, and plates were sealed and stored at −80° C.

Plasmid DNA isolation and DNA sequencing.

Plasmid DNA for sequencing template was isolated either by the Qiagen R.E.A.L. Prep 96 plasmid kit according to the manufacturer's protocol or by a solid-phase reversible immobilization method modified from an earlier published procedure (Hawkins, et al. 1994. Nucleic Acids Res. 22:4543-4544) for use on an integrated automation platform (ELVIS; see http://bioinfo.pbi.nrc.ca/robotics). For the robotic plasmid preparation, recombinant clones were cultured in 1.2 ml of Terrific Broth in deep-well (2 ml) 96-channel microtiter plates, pelleted by centrifugation, and lysed by an alkaline-SDS procedure. Lysates were made up to 10% Polyethylene Glycol 8000 and 0.5 M NaCl prior to the addition of 200 μg of COOH-derivatized paramagnetic beads (Seradyn). The bead slurry mixture was incubated with shaking for 5 min, and the beads were subsequently fractionated over permanent magnets, washed in 50% ethanol, dried, and resuspended in double-distilled H₂O. Plasmid concentration was estimated by resolving plasmid preps on 1% agarose gels.

High-throughput DNA sequencing reactions were conducted in 384-well microtiter plate format, by using 100 to 300 ng of template DNA in combination with 5′-biotinylated T7 and M13RP sequencing primers, in a ⅓ volume Big Dye sequence reaction (PE Biosystems). Reactions were assembled by the robotic system described above and thermocycled according to the supplier's recommended protocol. Sequence extension reaction products were purified by addition of 10 μg of streptavidin-paramagnetic beads (M-280; Dynal Corporation), followed by fractionation over permanent magnets. Fractionated beads were resuspended in 12 μl of 50% deionized formamide and treated at 95° C. for 5 min prior to immobilization and transfer of up to 12 μl of the reaction product-containing supernatant to a fresh 384-well microtiter plate. Completed reactions were sealed and stored at −80° C. prior to resolution on a PE-3700 capillary sequencing device.

Sequence data assembly and analysis.

All sequence data assembly, analysis, and storage were done by software available from the Canadian Bioinformatics Resource (http://www.cbr.nrc.ca). Raw sequencing data were assembled into contigs (sets of overlapping segments of DNA) for each template by Pregap4 (version 1.1) and Gap4 (version 4.6) in the Staden software package (release 2000.0; J. Bonfield, K. Beal, M. Betts, M. Jordan, and R. Staden, 2000). Contig nucleotide and peptide sequences were compared to a database of approximately 1,000 cpn60 sequences (cpnDB) by BlastP and BlastN. Sequence data, template information, and Blast results were deposited in a MySQL database for data storage and further analysis. Sequence manipulations, such as format changes and amino acid translations were done by GCG (Wisconsin package, version 10.1 for Unix). Sequence alignments were done with ClustalW and viewed with GeneDoc.

Phylogenetic analysis was performed by programs in the PHYLIP software package. Specifically, alignments were sampled for bootstrap analysis by Seqboot, distances were calculated with the PAM option of Protdist (for peptide sequences) or the maximum-likelihood option of Dnadist. Dendrograms were constructed from distance data by using neighbor-joining by neighbor. Consensus trees were calculated by Consense, and branch lengths were superimposed on consensus trees by Fitch. Completed trees were viewed by TreeView.

In another embodiment, a mixed DNA template representing a complex microbial community was provided by extracting total DNA from piglet feces. From this template, a region of the cpn60 gene sequence was amplified using universal, degenerate primers. Four independently amplified DNA products were produced by application of two methods for DNA extraction combined with two annealing temperatures for PCR, 40° C. and 56° C. The amplified products were cloned independently to produce four libraries. High quality sequence data were obtained for 1125 clones that were randomly selected from the four libraries (278 from B40, 332 from B56, 293 from P40 and 222 from P56). Disregarding the flanking degenerate primer sequences, the cloned cpn60 gene region was 552, 555 or 558 nucleotides in length (184, 185 or 186 codons, respectively).

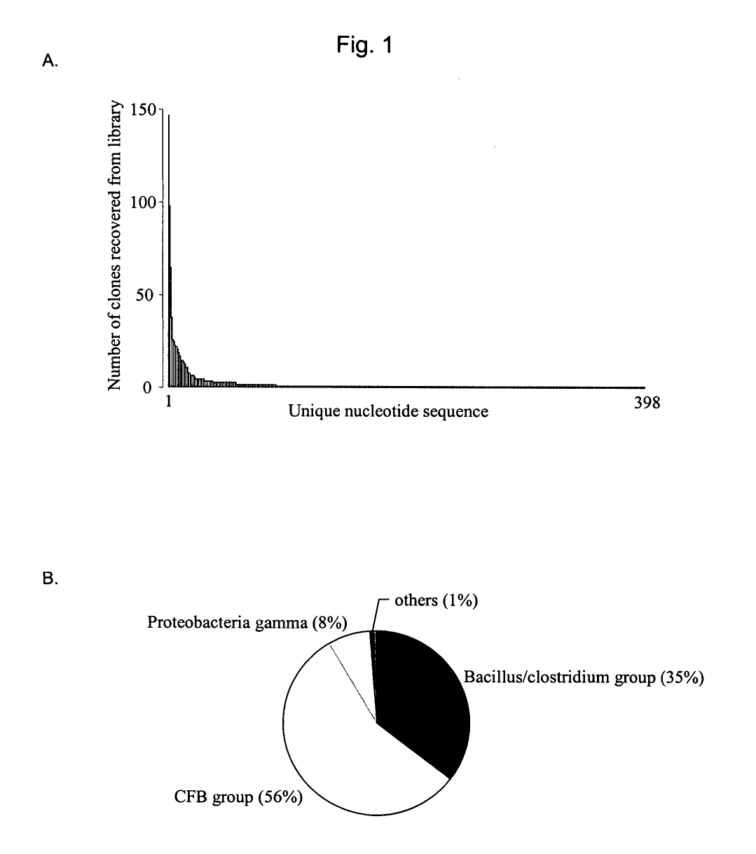

Pairwise comparisons of the 1125 sequences using CLUSTALw revealed the presence of 398 unique nucleotide sequences (encoding 280 unique peptide sequences). These were deposited in GenBank as a phylogenetic study and assigned the accession numbers AF436893-AF437290, wherein the accession numbers AF436893-AF437290 deposited in GenBank are incorporated herein in their entirety by this reference. FIG. 1A shows the number of times each unique nucleotide sequence was recovered from the total library. A few sequences were recovered frequently, one sequence 148 times; whereas 307 sequences were recovered only once. Only 10 sequences were recovered more than 20 times. Pairwise comparisons among the 398 unique sequences ranged from 47-99% nucleotide sequence identity.

Phylogenetic analysis of cpn60 sequence data.

Each DNA and peptide sequence was compared to a database of cpn60 sequences using BLAST. The database is a curated and growing collection of approximately 1100 eubacterial and eukaryotic cpn60 sequences harvested from public databases or generated in the laboratories of a network of collaborating researchers. The nearest database neighbors of the most frequently recovered library sequences are shown in Table 1. The estimated taxonomic breakdown of the total library contents, based on nearest neighbor taxonomy, is illustrated in FIG. 1B and Table 2. The largest taxonomic group, represented by 55% of the total library clones and 54% of the unique nucleotide sequences, was the cytophaga-flexibacter-bacteroides (CFB) group. The bacillus/clostridium subgroup of Gram-positive bacteria represented 36% of the total library clones and 42% of the unique nucleotide sequences and gamma-class Proteobacteria accounted for 8% of the total clones and 3% of the unique nucleotide sequences. The group labeled “others” in FIG. 1B included clones whose nearest database neighbors were in the spirochete, chlamydiales or Proteobacteria beta families (see Table 2 for details). Sequence length was strictly correlated with taxonomic assignment. That is, all clones with nearest neighbors in the CFB group had lengths of 558 bp (186 codons) whereas all the clones with nearest neighbors in the Proteobacteria gamma group and bacillus/clostridium group are 555 bp (185 codons) and 552 bp (184 codons), respectively. These are identical to the lengths observed for database reference sequences from each of these groups.

The most abundant sequence in the library (recovered 148 times), represented by clone 002_a03, is 88% identical at the amino acid level (78% nucleotide identity) to Prevotella intermedia ATCC25611. Other sequences recovered at least 4 times from the library are identified in Table 1 along with their nearest database neighbors. In three cases, library clones shared 100% DNA sequence identity with database reference strains: Lactobacillus amylovorus ATCC33620, Lactobacillus acidophilus T13 and Enterococcus asini ss-1501.

TABLE 1
Nearest cpn60 database neighbors of sequences recovered from library
at least four times out of 1125 clones.

| Clone | | GenBank | | % peptide | % DNA | |
|---|---|---|---|---|---|---|
| name | Nearest cpn60 database neighbor | accession | Taxonomic group | identity(similarity) | identity | Frequency |
| 002_a03 | Prevotella intermedia ATCC25611 | AF440234 | CFB group | 88(94) | 78 | 148* |
| 001_f12 | Prevotella bivia ATCC29303 | AF440233 | CFB group | 88(92) | 73 | 98* |
| 002_a11 | Anaerobiospirillum succiniciproducens ATCC700195 | AF441383 | Proteobacteria gamma | 86(94) | 80 | 65* |
| 001_c11 | Prevotella bivia ATCC29303 | AF440233 | CFB group | 88(92) | 72 | 38* |
| 001_e03 | Bacillus halodurans | AP001508 | Bacillus/clostridium group | 64(83) | 64 | 26* |
| 001_a02 | Clostridium thermocellum ncib10682 | Z68137 | Bacillus/clostridium group | 71(85) | 69 | 25* |
| 001_g07 | Clostridium thermocellum ncib10682 | Z68137 | Bacillus/clostridium group | 72(86) | 70 | 23* |
| 005_a04 | Prevotella bivia ATCC29303 | AF440233 | CFB group | 88(92) | 73 | 22* |
| 002_c12 | Bacteroides ovatus ATCC8483 | AF440236 | CFB group | 97(97) | 83 | 21* |
| 002_b08 | Clostridium difficile 79-685 | AF080547 | Bacillus/clostridium group | 73(88) | 63 | 19* |
| 003_b04 | Clostridium thermocellum ncib10682 | Z68137 | Bacillus/clostridium group | 72(86) | 70 | 17* |
| 001_a05 | Chryseobacterium gleum ATCC35910 | AF440235 | CFB group | 68(84) | 67 | 15* |
| 002_g03 | Thermoanaerobacter brockii Rt8.G4 | U56021 | Bacillus/clostridium group | 75(89) | 68 | 15* |
| 002_e03 | Clostridium thermocellum ncib10682 | Z68137 | Bacillus/clostridium group | 71(85) | 70 | 14* |
| 005_b04 | Prevotella bivia ATCC29303 | AF440233 | CFB group | 88(92) | 73 | 13* |
| 002_e11 | Bacteroides ovatus ATCC8483 | AF440236 | CFB group | 96(96) | 80 | 11* |
| 003_a04 | Anaerobiospirillum succiniciproducens ATCC700195 | AF441383 | Proteobacteria gamma | 83(91) | 79 | 11 |
| 005_c01 | Clostridium thermocellum ncib10682 | Z68137 | Bacillus/clostridium group | 74(88) | 71 | 8* |
| 005_e05 | Clostridium thermocellum ncib10682 | Z68137 | Bacillus/clostridium group | 72(88) | 72 | 8* |
| 001_h02 | Clostridium perfringens | X62914 | Bacillus/clostridium group | 71(88) | 66 | 7* |
| 003_f04 | Prevotella bivia ATCC29303 | AF440233 | CFB group | 88(93) | 74 | 7* |
| 008_h10 | Clostridium difficile 79-685 | AF080547 | Bacillus/clostridium group | 73(89) | 64 | 7* |
| 002_c10 | Prevotella bivia ATCC29303 | AF080547 | CFB group | 87(93) | 77 | 6* |
| 001 _h09 | Lactobacillus amylovorous ATCC33620 | | Bacillus/clostridium group | 100(100) | 100 | 5 |
| 001_h11 | Bacillus halodurans | AP001508 | Bacillus/clostridium group | 64(83) | 64 | 5* |
| 003_b12 | Pediococcus pentosaceus ATCC43200 | | Bacillus/clostridium group | 95(96) | 84 | 5* |
| 003_d12 | Clostridium thermocellum ncib10682 | Z68137 | Bacillus/clostridium group | 72(86) | 70 | 5 |
| 005_e02 | Prevotella intermedia ATCC25611 | AF440234 | CFB group | 89(93) | 82 | 5 |
| 005_e06 | Clostridium thermocellum ncib10682 | Z68137 | Bacillus/clostridium group | 72(86) | 70 | 5 |
| 006_b07 | Clostridium perfringens | X62914 | Bacillus/clostridium group | 64(84) | 64 | 5* |
| 006_f02 | Clostridium thermocellum ncib10682 | Z68137 | Bacillus/clostridium group | 78(89) | 71 | 5* |
| 001 _e12 | Clostridium thermocellum ncib10682 | Z68137 | Bacillus/clostridium group | 74(88) | 71 | 4* |
| 002_d06 | Prevotella intermedia ATCC25611 | AF440234 | CFB group | 88(94) | 78 | 4 |
| 002_d12 | Clostridium thermocellum ncib10682 | Z68137 | Bacillus/clostridium group | 71(86) | 68 | 4 |
| 002_g10 | Prevotella intermedia ATCC25611 | AF440234 | CFB group | 88(94) | 78 | 4* |
| 011_c01 | Lactobacillus acidophilus T-13 | | Bacillus/clostridium group | 100(100) | 100 | 4* |
| 014_a12 | Clostridium perfringens | X62914 | Bacillus/clostridium group | 72(87) | 70 | 4 |
| 018_b06 | Lactococcus garvieae ATCC43921 | AF245674 | Bacillus/clostridium group | 66(86) | 63 | 4 |

*recovered from at least two libraries.

TABLE 2
Summary of all library clones classified by nearest cpn60 database neighbors.

| | | | Number of | Number | | |
|---|---|---|---|---|---|---|
| | | GenBank | unique | of | % DNA | % peptide |
| Nearest cpn60 Database Neighbor | Taxonomic group | accession | sequences | clones | identity | identity(similarity) |
| Bacteroides forsythus ATCC43037 | CFB group | AJ006516 | 21 | 25 | 69-75 | 76(86)-88(92) |
| Bacteroides ovatus ATCC8483 | | AF440236 | 29 | 68 | 66-83 | 69(81)-97(97) |
| Bacteroides vulgatus ATCC8482 | | AF440238 | 10 | 10 | 63-85 | 70(82)-94(95) |
| Chryseobacterium gleum ATCC35910 | | AF440235 | 3 | 17 | 67-68 | 68(84)-73(82) |
| Prevotella bivia ATCC29303 | | AF440233 | 87 | 274 | 72-79 | 84(89)-89(94) |
| Prevotella intermedia ATCC25611 | | AF440234 | 44 | 210 | 72-82 | 72(84)-93(96) |
| Prevotella nigrescens ATCC33563 | | AF441382 | 19 | 20 | 68-81 | 67(83)-89(93) |
| Bacillus coagulans CECT 12 | Bacillus/clostridium group | AF441379 | 5 | 6 | 68-70 | 72(87)-74(86) |
| Bacillus firmus CECT 14 | | AF441380 | 1 | 2 | 66 | 65(82) |
| Bacillus halodurans | | AP001508 | 11 | 42 | 63-67 | 64(84)-71(86) |
| Bacillus psychrophilus CECT 4073 | | AF441381 | 8 | 9 | 66-70 | 72(86)-73(88) |
| Bacillus sp. MS | | AB028452 | 3 | 3 | 68-69 | 72(87) |
| Clostridium acetobutylicum | | M74572 | 2 | 3 | 66-68 | 65(84)-73(87) |
| Clostridium difficile 79-685 | | AF080547 | 14 | 42 | 61-65 | 64(78)-74(89) |
| Clostridium perfringens | | X62914 | 12 | 28 | 63-70 | 63(83)-73(88) |
| Clostridirum thermocellum ncib10682 | | Z68137 | 88 | 210 | 65-75 | 67(83)-82(91) |
| Enterococcus asini ss-1501 | | AF245671 | 7 | 11 | 59-100 | 71(85)-100(100) |
| Globicatella sanguinis ATCC51173 | | AF441384 | 1 | 1 | 62 | 63(83) |
| Lactobacillus acidophilus T-13 | | | 2 | 5 | 99-100 | 99(99)-100(100) |
| Lactobacillus amylovorous ATCC33620 | | | 3 | 9 | 95-100 | 97(99)-100(100) |
| Lactobacillus jensenii ATCC25258 | | | 1 | 1 | 64 | 66(83) |
| Lactococcus garvieae ATCC43921 | | AF245674 | 1 | 4 | 63 | 66(86) |
| Pediococcus pentosaceus ATCC43200 | | | 5 | 10 | 84-98 | 95(96)-100(100) |
| Thermoanaerobacter brockii Rt8.G4 | | U56021 | 2 | 16 | 68 | 75(89) |
| Anaerobiospirillum succiniciproducens | Proteobacteria gamma | AF441383 | 12 | 88 | 79-80 | 83(91)-86(94) |
| ATCC700195 | | | | | | |
| Burkholderia vietnamiensis DSM 11319 | Proteobacteria beta | AF104908 | 1 | 1 | 82 | 87(93) |
| | chlamydiales | NP_296764 | 1 | 2 | 55 | 50(68) |
| Chlamydia muridarum | | | | | | |
| Borrelia burgdorferi | spirochetes | NC_001318 | 3 | 5 | 62-67 | 65(82)-67(83) |
| Treponema pallidum | | AE001188 | 1 | 1 | 64 | 71(89) |
| | | TOTAL | 398 | 1125 | | |

Another clone shared 100% amino acid sequence identity and 98% nucleotide sequence identity with Pediococcus pentocaceus ATCC43200. Overall, the level of sequence identity between each of the 398 unique library sequences and its nearest database neighbor ranged from 56-100% DNA identity (51-100% peptide identity, 71-100% peptide similarity) with only 2 clones having less than 60% peptide identity to their nearest database neighbor. Table 2 shows the overall composition of recovered sequences in terms of their nearest database neighbors.

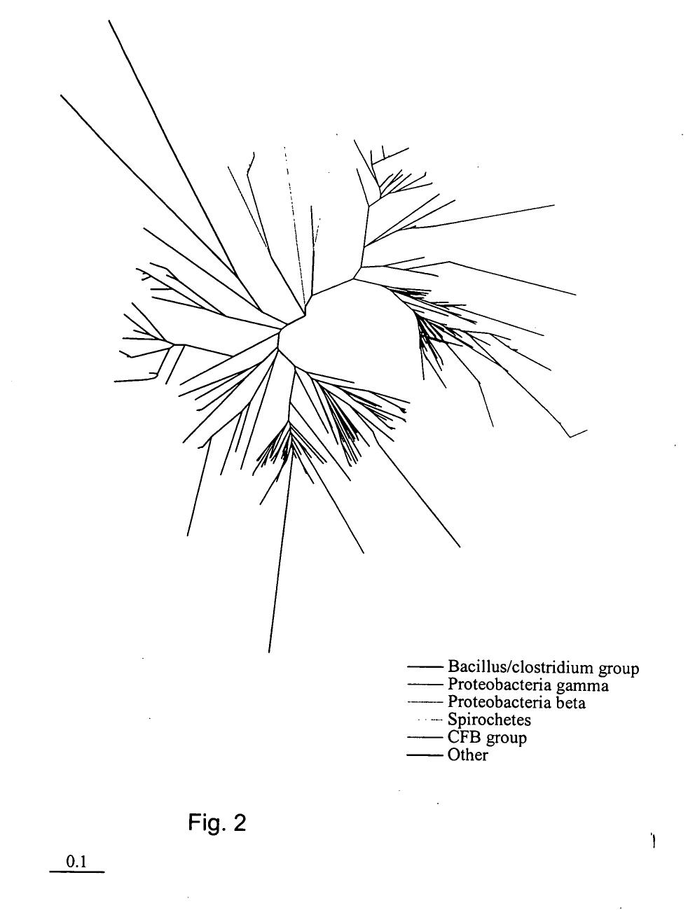

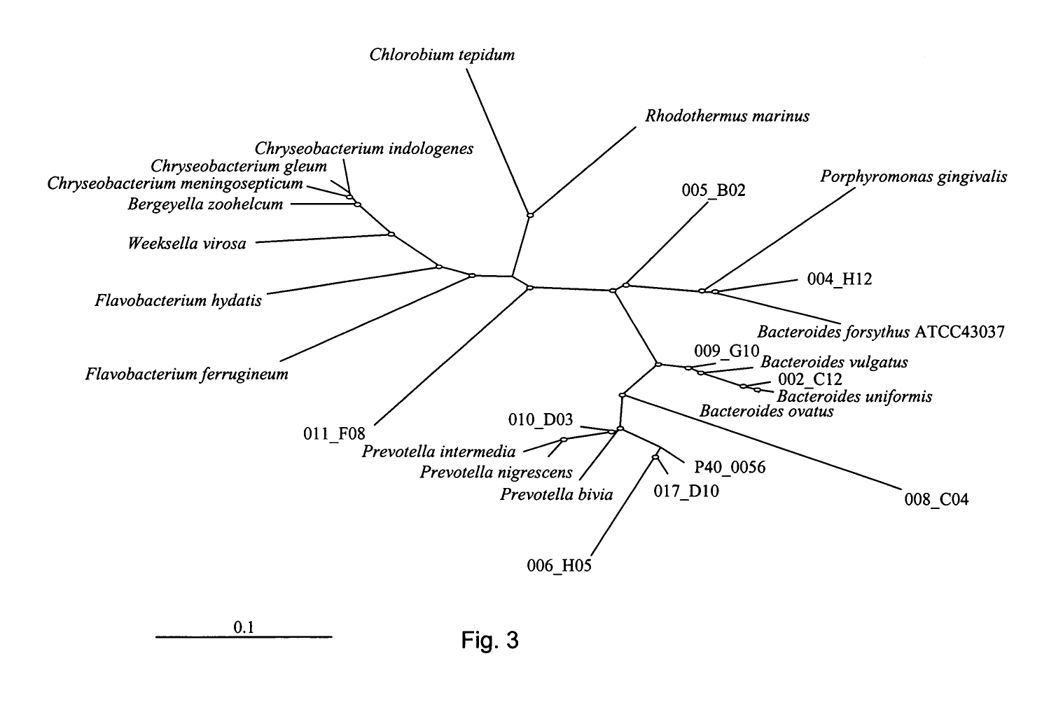

Following the gross phylogenetic analysis presented in FIG. 2, groups of cloned sequences from each of the represented taxonomic categories were selected for detailed phylogenetic tree construction, incorporating reference sequences from the cpn60 database. Using this technique, clone sequences were tentatively identified to the level of taxonomic subclass, family or genus. An example of this analysis, including ten clone sequences with nearest database neighbors in the CFB group and reference sequences from the genera Chlorobium, Rhodothermus, Flavobacterium, Bergeyella, Chryseobacterium, Bacteroides and Weeksella is shown in FIG. 3.

Genetic Diversity of Sampled Microorganisms.

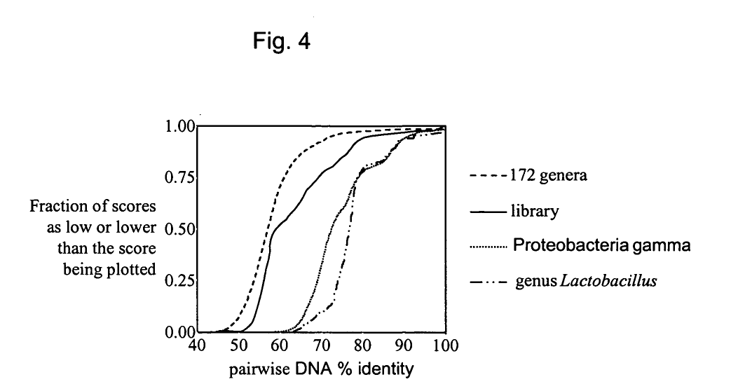

The cumulative frequency distribution (CFD) was plotted for the DNA sequence identity scores from all pairwise comparisons of library clone sequences (FIG. 4). To produce plots for comparison to this CFD plot of our experimental population, three other populations of cpn60 sequences were synthesized by selecting sequences from our database of cpn60 reference sequences. The first of these populations included individual species from 172 different genera (including both prokaryotes and eukaryotes) represented in the database. A second population was constructed by pooling cpn60 universal target sequences from 77 species (34 genera) of Proteobacteria gamma. The third population includes 37 species from a single genus, Lactobacillus. The experimental, pig feces library population, while less diverse than the population of 172 genera, was more diverse than the genus Lactobacillus, or the Proteobacteria gamma taxon, with approximately half of the pairwise comparisons within the library having DNA identities of 60% or less.

Sequence accuracy and microheterogeneity.

Clusters of nearly identical clone sequences (98-99% nucleotide identity) ranging in size from 2 to 20 sequences (191 total sequences) were further analyzed to determine the nature of the differences between the sequences. Multiple alignments of these groups of sequences showed that a disproportionate (p<0.001) number of the differences within the alignments were synonymous changes, occurring in the third position of codons. Examination of a total of 320 differences revealed that 61 were in the first position of codons, 63 were in the second position and 196 were in the third position. Almost all third position differences (191/196) were synonymous changes in terms of their effects on the encoded peptide sequence. No in-frame stop codons were observed in any of the 1125 clone sequences determined. Also, 29 of the 38 sequences in Table 1 (sequences occurring at least 4 times) were recovered from at least two of the four libraries.

DNA extraction methods and PCR conditions used affect organisms sampled.

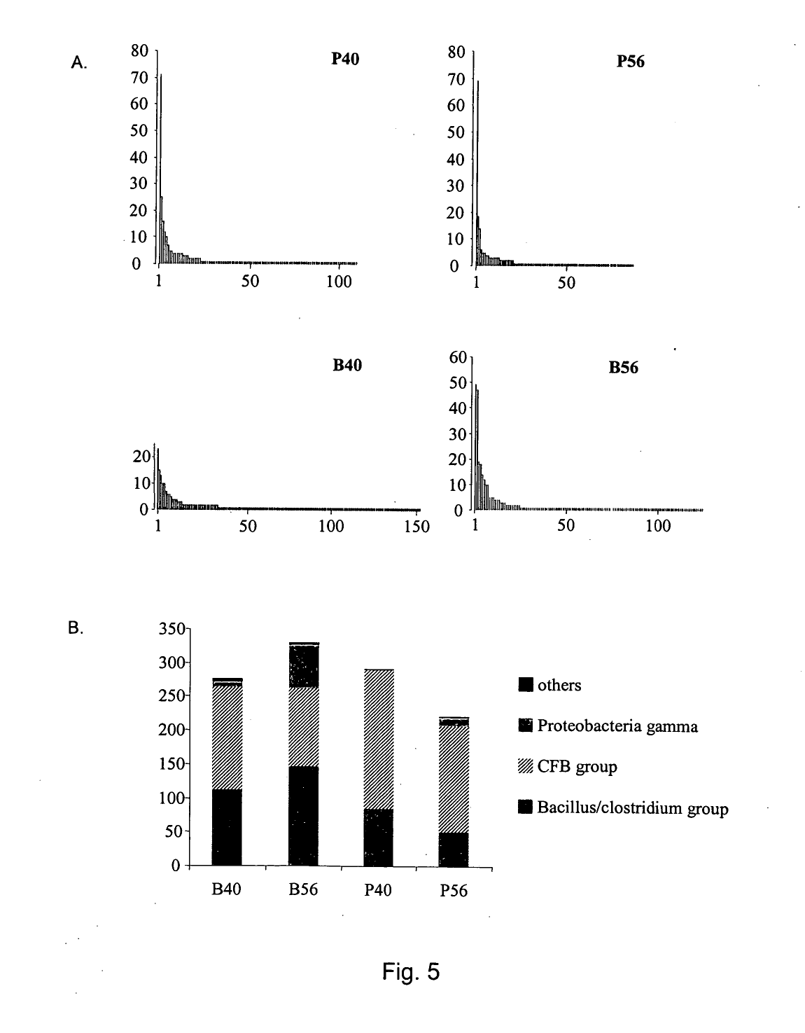

To assess the effects of library construction parameters on library contents, sequence data were grouped by library of origin and clone frequencies and taxonomic distributions were analyzed for each of the 4 data sets (FIG. 5A). The B40, B56, P40 and P56 libraries contained 156, 125, 112 and 91 different sequences, respectively. The frequency distributions of unique sequences varied markedly between libraries. While the most prevalent clone in the P56 and P40 libraries was recovered approximately 70 times from each library (accounting for 25-30% of clones sequenced) and the most prevalent clone in the B56 library was recovered 49 times (15% of clones), the most abundant clone in the B40 library was recovered only 23 times (8% of clones). FIG. 5B shows the taxonomic composition of each of the four libraries. The relative proportions of each taxon varied between the four libraries, with the largest proportion of Proteobacteria gamma-like clones occurring in the B56 library while this taxon was completely absent from the P40 library.

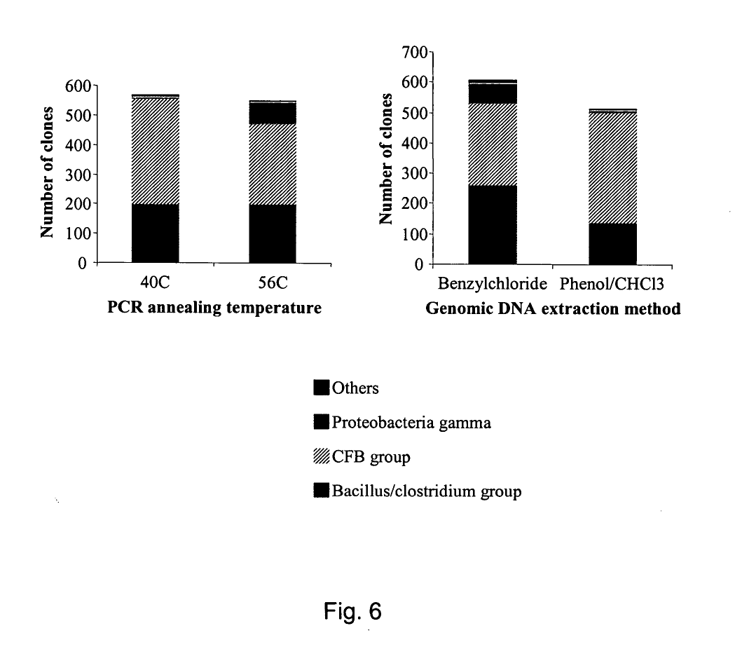

Data were also grouped for analysis according to the PCR annealing temperature or genomic DNA extraction method used in library construction. FIG. 6 shows the taxonomic composition of clones produced with a PCR annealing temperature of 40° C. versus 56° C. and DNA template prepared by the benzylchloride versus phenol:chloroform extraction methods. While all 4 taxonomic subclasses (CFB group, bacillus/clostridium group, Proteobacteria gamma and others) were detected in each group, the relative proportions of each taxon present varied with library construction conditions. For example, the highest proportion of Proteobacteria gamma class clones were produced with a PCR annealing temperature of 56° C. and a benzylchloride-extracted template (see also FIG. 5A).

Discussion.

The microbial community present in the gastrointestinal tract is complex and dynamic, varying in composition with age, diet, stress, medication, temperature, and varying along the length of the tract. Despite its importance, the microbial flora of the animal gut remains poorly characterized. In fact, microbial communities in general are poorly understood. The technologies developed for genomics programs present us with an unprecedented opportunity to advance our understanding of these populations. The present invention, which combined high throughput genomics technologies with an existing cpn60-based molecular diagnostic to characterize the pig feces microbial community, was undertaken as a feasibility study for the general application of this approach to other complex microbial communities.

Sequence data reveal biologically based microheterogeneity.

The degenerate PCR primers used in this study were previously demonstrated to amplify the universal target region of the cpn60 gene from a wide variety of organisms including eubacteria, fungi, plants and animals. The region of template-specific cpn60 amplified from pig feces total DNA varied in length, being either 552, 555 or 558 bp (184, 185 or 186 codons), and the complete sequence of each cloned PCR product was determined with two sequencing reactions initiated from sites within the cloning vector. Only unambiguous full-length sequences were included in our analysis.

To assess the potential impact of sequence artifacts that might have been introduced by PCR or Taq polymerase infidelity, clusters of nearly identical nucleotide sequences were examined. If the observed microheterogeneity in these sequence groups resulted from PCR-generated errors, then the distribution of sequence differences should be distributed uniformly among first, second and third positions within codons. It was observed, however, that a significantly disproportionate number of nucleotide differences occurred in the third codon position and that virtually all of these were synonymous differences, resulting in no change in the encoded peptide sequence. Also significant was the fact that no in-frame stop codons were observed in any of the clone sequences assembled. The genetic code includes 18 codons that with a single nucleotide change can be converted to a stop codon and analysis of the 1125 clone sequences indicated that there were 58,881 codons that were vulnerable to single nucleotide mutation to a nonsense codon. Thus, it is suggested that while PCR artifacts cannot be ruled out entirely, much of the minor sequence variation observed within clusters of related clone sequences was a reflection of real biological diversity. The sequence differences observed in many of these clusters was typical of the sorts of differences observed previously between cpn60universal target sequences from serotypes of a single bacterial species, Streptococcus suis.

A major concern in 16S rRNA sequence-based studies of microbial communities is the occurrence of chimeric PCR products. The generation of chimeric PCR products through template switching is facilitated by the presence of a number of highly conserved stretches along the primary structure of rDNAs and can involve closely related sequences, including multiple copies of 16S rRNA genes within a single genome. Chimeric cpn60 PCR products may be less likely to occur than 16S rRNA chimeras since cpn60 is present in fewer copies per genome (i.e., a single copy of cpn60 is present in most prokaryotic genomes), the amplified sequence is shorter (552-558 bp versus the approximately 1.6 kb amplified from bacterial 16S rRNA genes), providing fewer opportunities for template switching, and the cpn60 sequence lacks the intermittent highly conserved sequence stretches present in 16S rRNA genes. Chimera formation may be more likely, and difficult to detect if it occurred in-frame, between closely related cpn60 sequences. The best evidence that a sequence is not a PCR artifact is that it is recovered from more than one library, since the libraries were generated from independent PCR reactions.

In the present study, 29 of the 38 most frequently recovered sequences were recovered from at least two libraries. Also, several examples of pairs of very similar sequences independently recovered from at least two libraries were found. For example, the sequences of clones 001_a02 and 001_g07 are 98% identical and were each independently recovered from 3 of the 4 libraries. The same is true of 002_a03 and 002_g10 (99% identical, each recovered from 4 and 2 libraries, respectively) and 001_f12, 005_a04, 005_b04 and 001_c11 which have pairwise identities of 98% and were each recovered from b40 and p40 libraries. These examples provide evidence of real microheterogeneity in the population. The best way to systematically detect PCR chimeras in cpn60 libraries will be to apply computational tools such as CHECK_CHIMERA, developed for use in 16S rRNA-based studies.

The ability to track sequence microheterogeneity in complex microbial communities may have implications for our ability to understand the dynamics of these populations, particularly with respect to microbial evolution and concepts such as lateral gene transfer. Subtle sequence variation, typical of collections of sequences from closely related organisms, is overlooked in population profiling methods such as DGGE which rely on gross sequence attributes, or cloning and sequencing methods where sequences are grouped into general operational taxonomic units based on RFLP before individuals representative of each unit are sequenced.

Authors of previous studies of porcine fecal microflora have reported that the most predominant bacterial species outnumbers the next most abundant species by at least an order of magnitude and that culturable organisms were retrieved at frequencies that varied over many orders of magnitude. For example, while Bacteroidaceae have been found at 10¹⁰ cells per gram of feces and Bifidobacterium sp. at 10⁹ per gram of feces, less abundant organisms, such as E. coli have been reported at only 10⁵ per gram of adult pig feces. Based on these observations, it might be predicted that a study involving PCR amplification, cloning and sequencing of target genes from DNA extracted from such a population, would yield very little sequence diversity and that only the most abundant organisms would be represented. Instead, a great deal of sequence diversity was observed.

Out of 1125 clones, 398 distinct nucleotide sequences were observed, varying in frequency from 148 to 1, only two orders of magnitude. A likely explanation for the discrepancy between what was predicted and what was observed is the “Cₒt effect” (26). In a PCR reaction involving mixed template molecules, the amplification of the most abundant templates declines more rapidly during amplification cycles than that of less abundant templates due to the tendency for abundant templates and amplified products to re-anneal rather than undergo primer-mediated amplification. Thus, over the course of the PCR reaction, there is a normalization such that abundant templates become underrepresented and rare templates become overrepresented. While this phenomenon presents a serious challenge to experimental design strategies aimed at quantitating templates in the original sample, it works in favor of sampling maximum diversity.

The extent of sequence diversity found in the clone library is illustrated in FIG. 4 in a cumulative frequency distribution plot of the 79,003 pairwise DNA sequence comparisons derived from the 398 unique clone nucleotide sequences. When compared to populations constructed from a single genus, a taxonomic subclass and single representatives of 172 eukaryotic and eubacterial genera, the pig feces library falls between the taxonomic subclass distribution and the 172 genera population. FIG. 4 also shows that the pig feces library population plot is not a smooth curve like the 172 genera plot indicating the presence of clusters of various sizes of closely related sequences in the population as opposed to the uniform heterogeneity of the artificially constructed 172 genera population.

Comparison of experimental population to known fecal organisms.

Although some limited descriptive analysis of microbial populations resident in various gut compartments of pigs has been conducted (34,39), the majority of studies of gut microflora have used feces as a starting material. It is well established that the composition of fecal microbial populations varies widely from birth to adulthood as well as with diet changes and various disease states. Although there is tremendous variation in the proportions of gross taxonomic groups of organisms reported due to the different methods of isolation and characterization, there is some agreement on the types of organisms that comprise the normal porcine fecal flora.

As the fecal flora changes in composition from birth to maturity, there is an increase in the proportion of anaerobes and facultative anaerobes. Culture-based studies suggest that with maturity, CFB group organisms become dominant, particularly Bacteroides, with much smaller proportions of coliforms and Lactobacillus species and highly variable populations of Clostridium species being present. Greater phylogenetic and taxonomic detail is available for human fecal populations, which have been the focus of more molecular characterization and are thought to be somewhat similar to microbial populations in pig feces. In their analysis of PCR amplified and cloned 16S rRNA sequences from human feces, Suau et al. found that 95% of the 284 cloned sequences were related to CFB group organisms (particularly Bacteroides species) and Clostridium species. The taxonomic subclasses identified in this study, CFB group Bacillus/clostridium group and gamma class Proteobacteria, are consistent with these previous studies, as are the proportions identified (55% CFB group, 36% Bacillus/clostridium group and 8% gamma class Proteobacteria).

The goal of the current study was to identify and characterize taxonomically diverse organisms within the population. However, despite the potential normalizing effects of the PCR conditions, including the C₀t effect, the frequencies of sequences recovered in the library were most likely influenced by the frequencies of the source organisms in the population.

In addition to the three major taxonomic subclasses detected, our sequencing efforts also revealed relatively rare clone sequences assigned to the spirochete group and the beta class Proteobacteria. Our observation of clone sequences with similarity to the spirochete family is not unexpected since non-pathogenic spiral rods have been reported in microscopic observations of feces from healthy pigs and have been cultured from similar sources. The observation of a cloned sequence with 82% nucleotide identity and 87% peptide identity (93% similarity) to Burkholderia vietnamiensis, a member of the beta class of Proteobacteria, is interesting since members of this bacterial family have not been reported in studies of fecal flora from animals. However, members of the genus Burkholderia are known to include soil and rhizosphere bacteria as well as plant and human pathogens so perhaps it is not surprising that genomic DNA from this group of organisms would be present in pig feces.

Interestingly, no sequences with similarity to bifidobacteria were recovered. Bifidobacterium sp. are reportedly a major constituent of the fecal flora of monogastrics such as pigs and humans where they have been detected in culture-based studies at frequencies of 10⁹ cfu per gram of feces. It seems unlikely that the absence of bifidobacterial PCR products was due to failure of the PCR primers to anneal to these templates since previous work done in our laboratory, and available sequence data from a number of bifidobacterial cpn60 genes demonstrates that primer-binding sites are preserved in these organisms. It seems more likely that the genomic DNA preparation methods used failed to capture bifidobacteria DNA due a lack of sufficient mechanical force to break open these bacteria or that the relatively high G+C content of bifidobacterial sequences prevented efficient PCR amplification of these targets.

Failures to amplify and clone 16S rRNA sequences from Bifidobacterium sp. have also been reported in studies of human fecal flora. Recently, this issue was addressed by isolating total genomic DNA from a similar pig fecal sample using a bead-beating method and conducting PCR with Bifidobacterium-specific primers designed to amplify a 180 bp region within the cpn60 target. The resulting PCR products were cloned and when a small number of the clones were sequenced, they were found to be 99% identical at the DNA level to Bifidobacterium animalis. This result suggests that bifidobacteria are indeed present in pig feces and that their absence in the library sample is either the result of a failure to isolate the genomic DNA template with chemical methods or that the bifidobacterial template DNA is so rare in the template pool that it was not represented in the sample of 1125 clones.

Library construction methods affected library contents.

To assess the effects of library construction parameters on our results, four libraries were created using genomic DNA templates prepared by one of two methods and conducted the PCR reactions at either of two annealing temperatures. FIGS. 5 and 6 illustrate that these parameters did indeed have a pronounced effect on the contents of the resulting library. The Proteobacteria gamma group of templates seems particularly affected by template preparation method and PCR annealing temperature as indicated by a higher proportion of these sequences in libraries constructed from benzylchloride-extracted template DNA and higher annealing temperature.

Thus, while accounting for approximately 20% of clones in the B56 library, Proteobacteria gamma species were completely absent from the P40 library. Potential challenges for approaches such as those described herein include the possibility for systematic biases in the representativeness of templates within total DNA extracts compared to that of the original microbial population, and biases introduced by the degenerate primers and PCR conditions with respect to amplification from specific DNA templates. The small size of our initial fecal sample (approximately 2 g of feces from 5 pigs) may also be a factor in the representativeness of the genomic DNA extracts since feces are likely not homogenous and areas of concentration of some bacterial species may exist.

Cpn60 sequence database.

The clone with weak sequence similarity to Chlamydia muridarum (clone 007_D05) is indicative of current limitations to sequence identification (Table 2). The ability to assign cloned sequences to taxonomic subclass or beyond that to the level of genus or species is necessarily limited by the availability of relevant reference sequence data. The tree shown in FIG. 3 is a good illustration of both the strengths and weaknesses in our ability to identify clone sequences. While in some cases identification to the level of genus or even species is possible, there are other cases where limited reference data make it possible to identify sequences only to the level of taxonomic subclass. Currently, our database contains approximately 1100 reference sequences.

### Example 1B.

In this example, forty-five pigs (35 days of age) were fed diets containing corn (yellow dent), wheat (Laura) or barley (Brier) as the primary source of energy for 3 weeks. Pig diets were formulated to contain similar digestible energy and 3.15 g of digestible lysine per Mcal digestible energy. These diets did not contain any antibiotics. Pig body weight and feed intake were measured. At the end of the experiment, pigs were euthanized by CO₂ asphyxiation and exsanguinations, and their intestinal tracts were removed. Samples of digestive contents (digesta) were collected aseptically from the mid-ileum (75% of the distance between the duodenum and the ileo-caecal junction) and caecum. The numbers of total aerobes, total anaerobes, Enterobacteria, Lactobacillus spp., Clostridium spp. and Streptococcus spp. present in the digesta samples were enumerated as described (Estrada et al. 2001. Can.J.Anim.Sci. 81:141-148).

Total genomic DNA was isolated from 200 mg of ideal digesta using a combination of phenol:chloroform extraction and bead-beating. A pool of DNA from the 15 mammals in each diet group was created, resulting in 3 templates for PCR amplification. Each DNA pool was used as a template in 4 PCR reactions that differed only in the annealing temperature used. Previous studies indicated that amplifying samples over a range of annealing temperatures (42-56° C.) increases the diversity of the resulting pool of PCR products. The PCR products produced in each set of 4 reactions were pooled, agarose gel purified and ligated into vector pCR2.1-TOPO. Ligation mixtures were used to transform E. coli Top-10. The 3 resulting libraries (C (corn), B (barley) and W (wheat)) were allowed to propagate by plating on LB/ampicillin/X-Gal and 1248 white colonies were picked from each library. Colonies were cultured in 96-well plates (13 plates per library) and stored as glycerol stocks at −80° C. until sequencing template preparation. Plasmid purification, quantification and sequencing reaction assembly, thermocycling and resolution were done according to the methods described (Hill, et al. 2002. AppL. Environ. Microbiol. 68:3055-3066).

The total numbers of clones successfully sequenced (success is defined as a complete sequence of the full “universal target” with no sequence ambiguities) are summarized in Table 3. Over 900 sequences were obtained for each of the C, B and W libraries. The “universal target” refers to the sequence amplified from any cpn60 gene with the primers H279 and H280. In reference to E. coli, the universal target encompasses nucleotides 247-854 (including primer landing sites) or 274-828 (excluding degenerate primer landing sites). In general, the universal target (excluding degenerate primer landing sites) is 552, 555 or 558 bp in length wherein the length varies in a way consistent with the taxonomy of the subject, e.g., gamma class proteobacteria have 555 nt, low-GC gram positive bacteria have 552 and Bacteroides ssp. have 558.

TABLE 3
Number of sequences generated for each library.

| | | Number of unique |
|---|---|---|
| Library | Number of sequences | Sequences |
| C | 909 | 136 |
| B | 930 | 171 |
| W | 915 | 110 |
| C + B + W | 2751 | 316 |

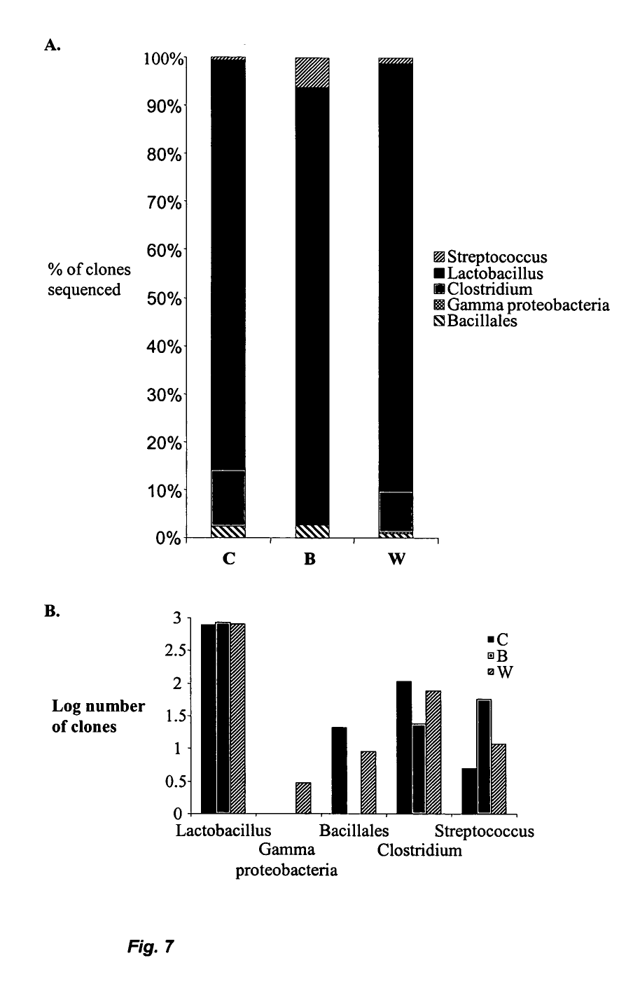

Following assembly and editing of the sequences, each sequence was compared to the cpnDB database using FASTA (for nucleotide sequences) or BLASTp (for peptide sequences). The nearest database neighbor for each clone was recorded. A graphical summary of the contents of each library based on the genus or taxonomic group of the nearest database neighbor for each clone is shown in FIG. 7. In general, all three libraries were dominated by Lactobacillus-like sequences, which constituted 84% of the C library, 92% of the B library and 90% of the W library.

Most of these Lactobacillus-like sequences were 95-100% identical to Lactobacillus amylovorus ATCC33620. In fact, sequences 90-100% identical to L. amylovorous ATCC33620 account for 1399 of the 2751 clones sequenced. Clostridium-like sequences were more abundant in the C library (12% of clones sequenced) as compared to the B library (2% of clones) or the W library (8%). Another difference between the libraries was the relative prevalence of Streptococcus-like sequences in the B library (6% of clones) compared to the C and W libraries (1% of each). These Streptococcus-like clones included sequences identical to Streptococcus orisratti, S. thermophilus and S. alactolyticus.

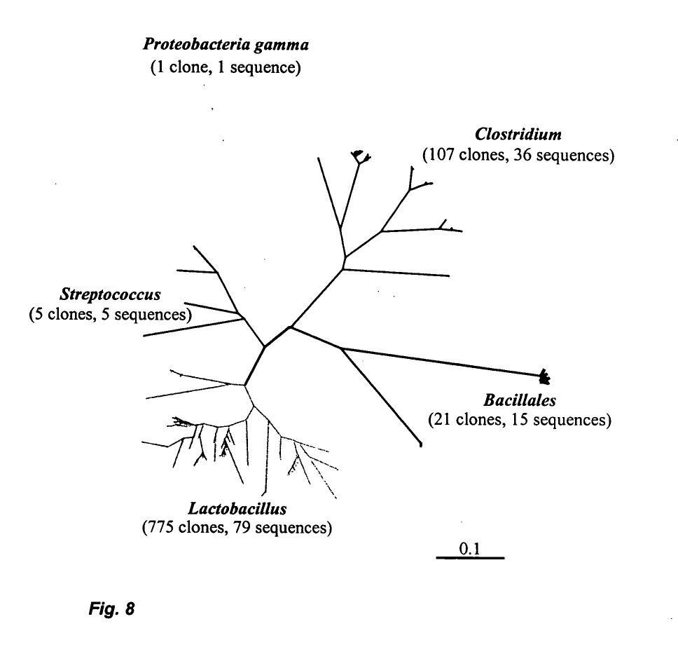

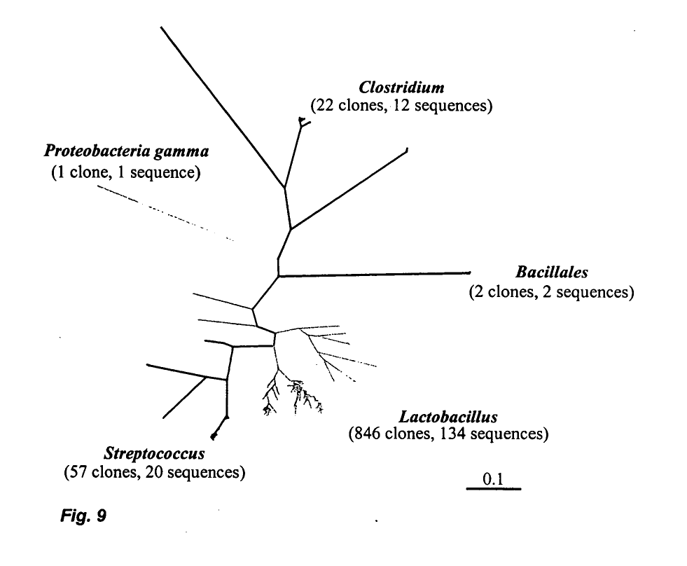

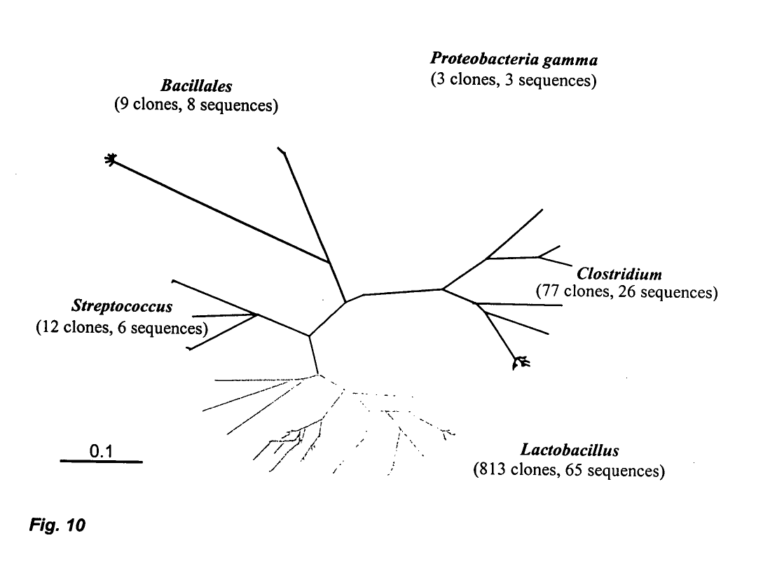

Phylogenetic analyses are shown in FIG. 8, FIG. 9 and FIG. 10. The contents of the three libraries were compared to determine the degree of overlap in sequences recovered. Table 4 summarizes the overlaps determined. Most of the B-specific sequences were found within the Lactobacillus-like and Streptococcus-like sequence groups while most of the C-specific sequences were found in the Clostridium-like groups. W-specific sequences were more evenly distributed across all sequence groups.

TABLE 4
Summary of library overlap.

| Library | Number of sequences |
|---|---|
| C only | 76 |
| B only | 127 |
| W only | 51 |
| C and B | 10 |
| C and W | 22 |
| B and W | 8 |
| C and B and W | 22 |
| TOTAL | 316 |

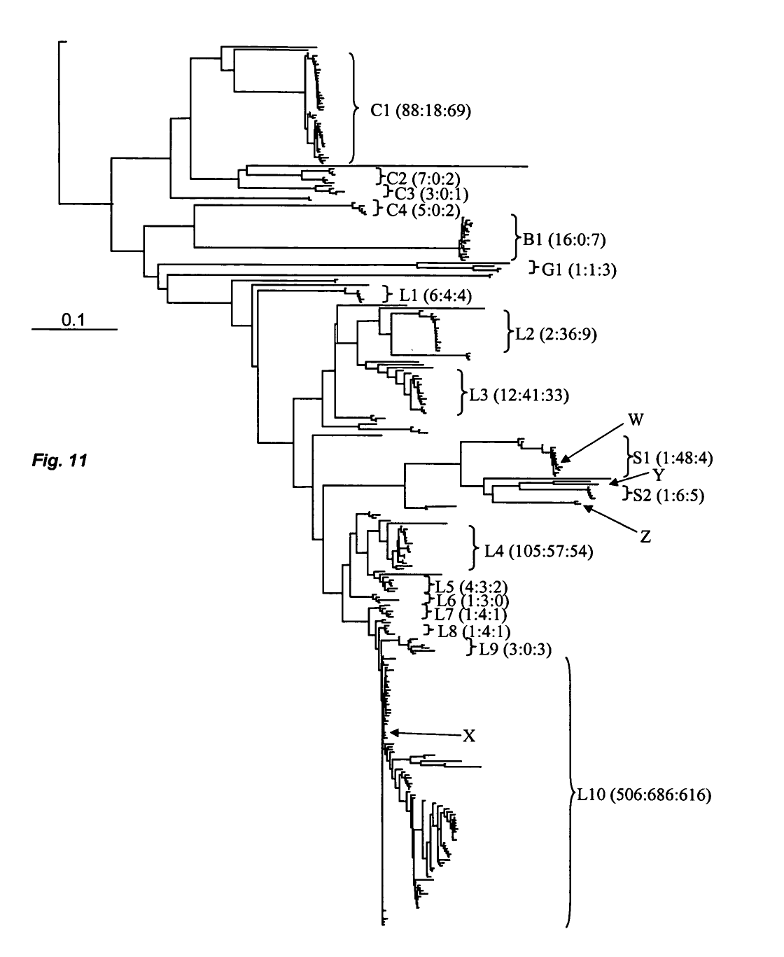

A phylogenetic analysis of the 316 unique nucleotide sequences found in the pooled C, B and W libraries is shown in FIG. 11. This analysis was used to identify clusters of closely related sequences within the pooled libraries and to calculate the numbers of clones in each group recovered from each library. This calculation resulted in a “C:B:W” ratio for each sequence group. For example, group C1, which includes a closely related group of Clostridium-like sequences, has a C:B:W ratio of 88:18:69, indicating that sequences falling into this group were recovered 88 times from the C library, 18 times from the B library and 69 times from the W library.

The relative proportions of Lactobacillus, Clostridium, Streptococcus and Proteobacteria-gamma-like sequences are consistent with those reported for the culture-based assessment of the ileum populations where lactobacilli were found to be present at 10⁸ cfu/g starting material, clostridia at 10⁷ cfu/g, streptococci at 10⁶ cfu/g and Enterobacteria (Proteobacteria gamma) at 10⁵ to 10⁶ cfu/g depending on the library.

In the enumeration study, the barley diet was associated with decreased numbers of Enterobacteria while the barley and wheat diets were associated with an increase in the number of lactobacilli. These patterns were not observed in the library sequencing study. Numbers of Lactobacillus-like sequences recovered from each library were approximately equal and too few Enterobacterial (gamma proteobacteria) sequences were recovered to draw any conclusions about diet-associated effects on this taxon. Sequence data indicated a relative decrease in the frequency of Clostridium-like and bacillus-like sequences and a corresponding increase in Streptococcus-like sequences associated with the barley diet. Comparisons of culture data and sequence data are naturally problematic since the criteria used to assign organisms or sequences to a given taxon are different.

Groups C1, B1 and S1 have C:B:W ratios of 88:18:69, 16:0:7 and 1:48:4, respectively. These groups were chosen as targets for the design of group-specific PCR primers and probes for real-time PCR. Using the SignatureOligo software (LifeIntel, Inc.), PCR primers were designed which would amplify sequences from the target group, but not any other sequences in the C, B or W libraries or any other sequences in cpnDB. Primer and probe sequences and predicted product sizes are outlined in Table 5. The specificity of each primer set was verified by testing the primer set against a range of templates including clones derived from the target group as well as neighboring groups.

TABLE 5

Primers and probes for real-time PCR of sequence groups B1, C1
and S1.

| | | | Product |
|---|---|---|---|
| Target | Primer sequences | Probe sequence | size |
| B1 | forward 5′-TGCAGGAGCAAATCCAATGAT-3′ | NA | 173 |
| | (SEQ ID NO: 3) | | |
| | reverse 5′-GCATGGCTTCGGCAATTAAA-3′ | | |
| | (SEQ ID NO: 4) | | |
| C1 | forward 5′-GCTGTTGATGTAGCAGTTGA-3′ | 5′-TGTTGCTGCGG | 155 |
| | (SEQ ID NO: 5) | GCATGAACC-3′ | |
| | | (SEQ ID NO: 6) | |
| | reverse 5′-ATAACCCCTTCGTTTCCTAC-3′ | | |
| | (SEQ ID NO: 7) | | |
| S1 | forward 5′-AACTTGACGTGGTTGAAGGG-3′ | NA | 172 |
| | (SEQ ID NO: 8) | | |
| | reverse 5′-GTTTTCAAGACTTCTTCAAGCAA-3′ | | |
| | (SEQ ID NO: 9) | | |

The C1 primers were used in quantitative PCR reactions with SYBR-green. In initial experiments, the threshold cycle (Cₜ) values for C1 group templates (clones w13_f01 and b10_c11) were determined to be 8.7 and 7.7, respectively, which are significantly lower than the Cₜ values determined for template derived from other taxonomic groups: c08_h12 from the B1 group (Cₜ=21.7, mean of 2 experiments) and b03_a08 from L1 (Cₜ=32.6, mean of two experiments). Results of SYBR-green PCR using C1 primers and the C, B and W genomic DNA templates are shown in Table 6.

TABLE 6
SYBR-green results for C1 primers on genomic DNA from
ileum contents of pigs on corn (C), barley (B) or wheat (W) diets.

| | | | | Number of |
|---|---|---|---|---|
| | | | | clones |
| Template DNA | Cₜ (expt. 1) | Cₜ(expt. 2) | Mean Cₜ | recovered |
| C | 20.8 | 20.7 | 20.75 | 88 |
| B | 24.2 | 23.8 | 24.0 | 18 |
| W | 23.2 | 22.9 | 23.05 | 69 |

### Example 2.

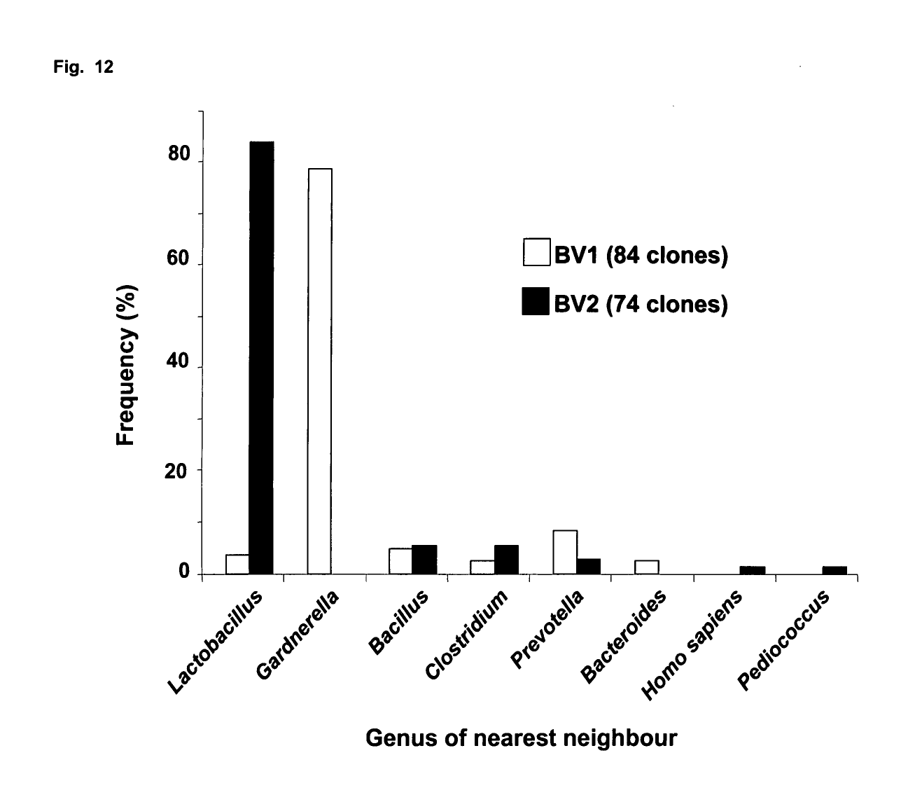

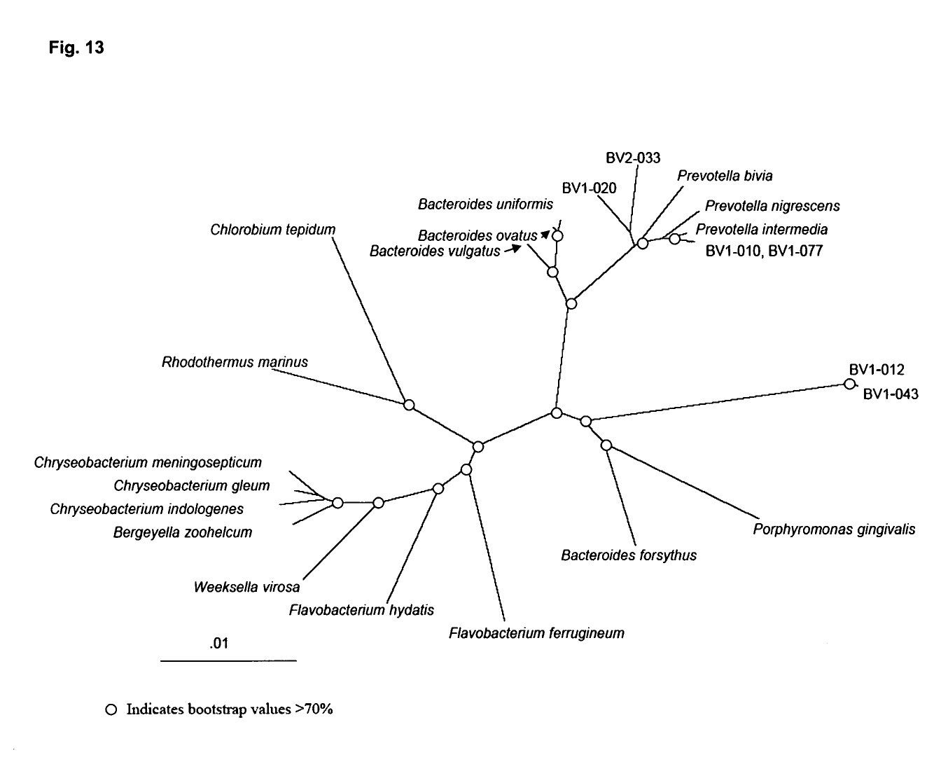

Vaginal swab samples were obtained from two individual human subjects. Using methods similar to those described in Example 1, total genomic DNA was isolated from the samples and subjected to universal cpn60 primer PCR to amplify partial cpn60 gene sequences. Libraries of partial cpn60 sequences were created by ligating the PCR products into cloning vectors. Ninety-six clones were randomly chosen from each library (BV1 and BV2). Sequencing of the isolated clones yielded 84 and 74 complete, unambiguous sequences from BV1 and BV2 libraries, respectively. Unique sequences within each library were identified (see SEQ ID NOs: 12 through 45) and frequencies of each of the sequences were calculated. Each unique sequence was compared to the cpnDB database and putative identifications were made (Table 7). Frequencies of various taxonomic groups were compared across the two libraries (FIG. 12) and detailed phylogenetic analysis of library constituents was performed to solidify their sequence-similarity-based identification (e.g., FIG. 13). Results of the analysis and inter-library comparison clearly demonstrate significant differences in the flora of the two individuals. Library BV1 is typical of healthy vaginal flora, wherein the contents of library BV2 are indicative of bacterial vaginosis.

TABLE 7
Putative identities and frequencies of sequences recovered from clone libraries BV1 and BV2.

| clone | nearest reference sequence | % dna identity | putative taxonomy | frequency |
|---|---|---|---|---|
| bv1-095 | Gardnerella vaginalis ATCC14018 | 99.275 | Bacteria; Gram +; Actinobacteria | 48 |
| bv1-075 | Gardnerella vaginalis ATCC14018 | 99.094 | Bacteria; Gram +; Actinobacteria | 6 |
| bv1-087 | Prevotella intermedia ATCC25611 | 84.74 | Bacteria; Other Prokaryotes; CFB group | 4 |
| bv1-099 | Gardnerella vaginalis ATCC14018 | 98.913 | Bacteria; Gram +; Actinobacteria | 3 |
| bv1-093 | Gardnerella vaginalis ATCC14018 | 99.094 | Bacteria; Gram +; Actinobacteria | 3 |
| bv1-090 | Enterococcus raffinosus 0286-86 | 72.232 | Bacteria; Gram +; Bacillus/clostridium group | 2 |
| bv1-069 | Gardnerella vaginalis ATCC14018 | 98.913 | Bacteria; Gram +; Actinobacteria | 2 |
| bv1-027 | Gardnerella vaginalis ATCC14018 | 98.913 | Bacteria; Gram +; Actinobacteria | 2 |
| bv1-080 | Lactobacillus acidophilus (API 92.0%) | 85.326 | Bacteria; Gram +; Bacillus/clostridium group | 2 |
| bv1-050 | Prevotella intermedia ATCC25611 | 77.738 | Bacteria; Other Prokaryotes; CFB group | 2 |
| bv1-072 | Bacillus mycoides CECT 4128 | 72.414 | Bacteria; Gram +; Bacillus/clostridium group | 1 |
| bv1-033 | Bacillus mycoides CECT 4128 | 72.595 | Bacteria; Gram +; Bacillus/clostridium group | 1 |
| bv1-012 | Bacteroides forsythus ATCC43037 | 69.3 | Bacteria; Other Prokaryotes; CFB group | 1 |
| bv1-082 | Clostridium acetobutylicum ATCC824 | 76.757 | Bacteria; Gram +; Bacillus/clostridium group | 1 |
| bv1-015 | Clostridium acetobutylicum ATCC824 | 76.937 | Bacteria; Gram +; Bacillus/clostridium group | 1 |
| bv1-096 | Gardnerella vaginalis ATCC14018 | 98.37 | Bacteria; Gram +; Actinobacteria | 1 |
| bv1-067 | Gardnerella vaginalis ATCC14018 | 99.271 | Bacteria; Gram +; Actinobacteria | 1 |
| bv1-061 | Gardnerella vaginalis ATCC14018 | 98.732 | Bacteria; Gram +; Actinobacteria | 1 |
| bv1-045 | Gardnerella vaginalis ATCC14018 | 98.007 | Bacteria; Gram +; Actinobacteria | 1 |
| bv1-004 | Gardnerella vaginalis ATCC14018 | 99.094 | Bacteria; Gram +; Actinobacteria | 1 |
| bv1-044 | Lactobacillus acidophilus (API 92.0%) | 84.964 | Bacteria; Gram +; Bacillus/clostridium group | 1 |
| bv1-077 | Prevotella intermedia ATCC25611 | 84.56 | Bacteria; Other Prokaryotes; CFB group | 1 |
| bv1-043 | Rhodothermus marinus ITI 376 | 70.909 | Bacteria; Other Prokaryotes; CFB group | 1 |
| bv2-089 | actobacillus acidophilus T-13 | 100 | Bacteria; Gram +; Bacillus/clostridium group | 50 |
| bv2-081 | Lactobacillus acidophilus T-13 | 99.819 | Bacteria; Gram +; Bacillus/clostridium group | 9 |
| bv2-086 | Bacillus mycoides CECT 4128 | 72.414 | Bacteria; Gram +; Bacillus/clostridium group | 4 |
| bv2-085 | Clostridium thermocellum ncib 10682 | 66.788 | Bacteria; Gram +; Bacillus/clostridium group | 3 |
| bv2-037 | Prevotella intermedia ATCC25611 | 77.778 | Bacteria; Other Prokaryotes; CFB group | 2 |
| bv2-057 | Clostridium thermocellum ncib 10682 | 66.788 | Bacteria; Gram +; Bacillus/clostridium group | 1 |
| bv2-032 | Homo sapiens mitochondrion | 94.054 | Eukarya; Animals; Mammals | 1 |
| bv2-068 | Lactobacillus acidophilus T-13 | 93.116 | Bacteria; Gram +; Bacillus/clostridium group | 1 |
| bv2-054 | Lactobacillus acidophilus T-13 | 99.819 | Bacteria; Gram +; Bacillus/clostridium group | 1 |
| bv2-023 | Lactobacillus acidophilus T-13 | 99.819 | Bacteria; Gram +; Bacillus/clostridium group | 1 |
| bv2-041 | Lactobacillus fermentum (API 93.7%) | 86.232 | Bacteria; Gram +; Bacillus/clostridium group | 1 |

### Example 3.

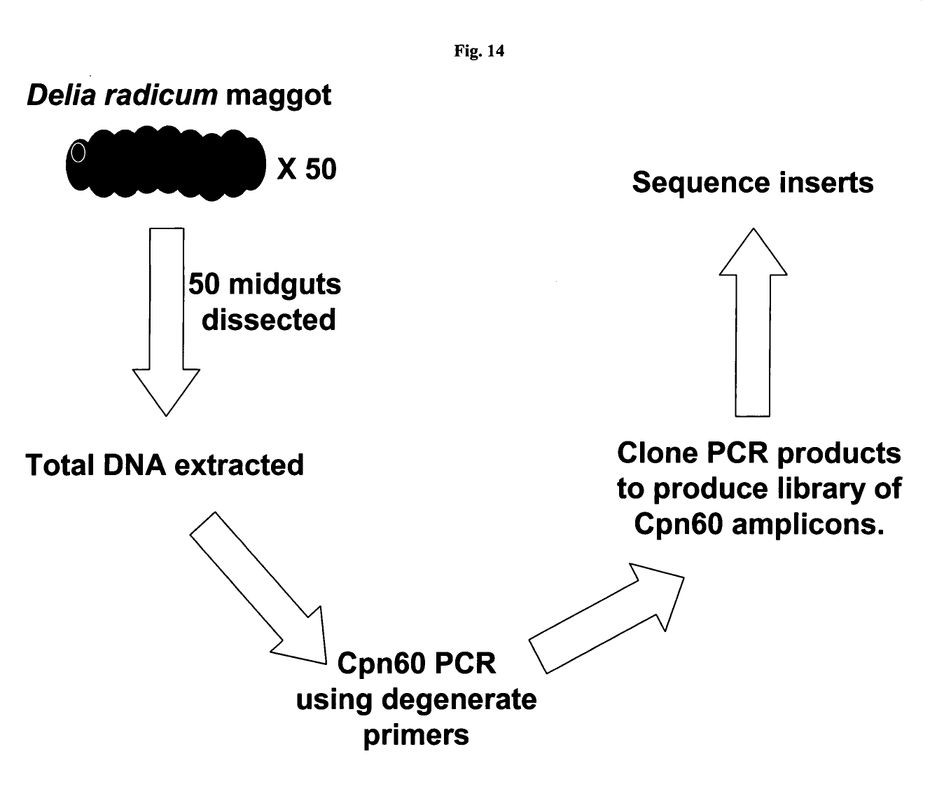

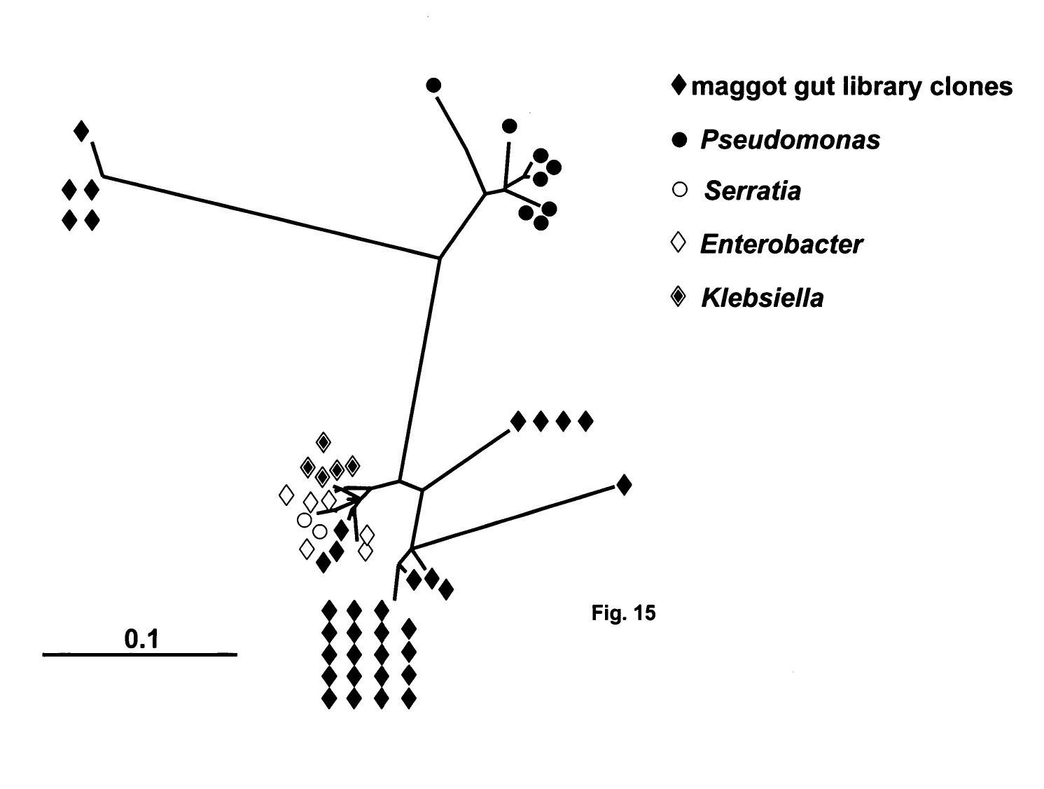

Using methods similar to those described in Example 1, midguts were dissected from 50 Delia radicum (crucifer root maggots) and total genomic DNA was isolated from these tissues. PCR was performed using the universal cpn60 primers and total genomic DNA as template. Libraries of partial cpn60 sequences were created by ligating the PCR products into cloning vectors. The experimental scheme for the creation of cpn60 sequence libraries from the midgut microflora of Delia radicum is depicted in FIG. 14. Thirty-six randomly selected clones were sequenced (see SEQ ID NOs: 46 through 58). Sequences were pooled according to identities. Representatives of each unique sequence were compared to the cpnDB database for identification. Phylogenetic analysis of the library sequence data was also performed (FIG. 15).

Example 4

In another embodiment of the invention, a method for designing primers for quantitative PCR is disclosed. The first step is the amplification of nucleic acid using universal primers and the creation of libraries of cpn60 clones for sequencing. The second step is the clustering of sequence data based on phylogenetic analysis, calculation of clone frequencies corresponding to each sequence cluster and the creation of the frequency ratio (like the C:B:W ratio in the pig ileum example of Example 1). The third step is the use of the information of step 2 for the rational design of taxon or cluster-specific primer sets for quantitative PCR.

Biopsy samples are obtained from 2 individuals—one individual with “x” disease and one “normal” or healthy individual. cpn60 sequence libraries are produced for both individuals as described herein. The sequence data is collected (1000 sequences per library). After clustering the data and calculating the frequency with which sequences in each sequence cluster occur, it is determined that the frequency ratio for Bacteroides-like sequences is 50:635 for “x”:normal. This indicates that the Bacteroides-like sequence cluster would be an excellent target for further investigation with respect to “x”. The sequences in that cluster are used to design primers which will specifically amplify these sequences and none of the other sequences in the library (or in the known universe of cpn60 sequences). Thus, there is provided a powerful tool for doing in vivo studies where one may quantitatively screen large numbers of biopsies or samples for this sequence cluster and demonstrate a link between the pathology and this taxon. Thus, there is provided a means of identifying taxons with potential links to pathologies.

1. A process for characterizing a community of organisms, the process comprising:
isolating nucleic acid from the community of organisms;
contacting the isolated nucleic acid with at least one primer, wherein the at least one primer is capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate; and
comparing the sequence of the amplificate to a database of sequences or to the sequence of other amplificates obtained from the community of organisms.

2. The process according to claim 1, further comprising performing a phylogenetic analysis on the sequence of the amplificate.

3. The process according to claim 1, further comprising depositing the sequence of the amplificate into a database of cpn60-like sequences.

4. The process according to claim 2, further comprising phylogenetically categorizing at least one organism within the community of organisms based on the sequence of the amplificate.

5. The process according to claim 4, further comprising designing an amplificate specific oligonucleotide capable of hybridizing to the sequence of the amplificate.

6. The process according to claim 5, further comprising quantitatively amplifying the isolated nucleic acid with the amplificate specific oligonucleotide.

7. The process according to claim 6, wherein the amplificate specific oligonucleotide is incapable of amplifying a sequence from another amplificate having a different sequence than that of the sequenced amplificate.

8. The process according to claim 6, further comprising:
calculating a frequency of the at least one organism within the community of organisms; and
comparing the frequency of the at least one organism in the community of organisms to a frequency of a known community of organisms.

9. The process according to claim 6, further comprising comparing an abundance of the at least one organism in the community of organisms to an abundance of the at least one organism in a known community of organisms.

10. The process according to claim 1, wherein the community of organisms is selected from the group consisting of intestinal flora, vaginal flora, midgut flora, biofilm flora, soil flora, Gram-positive flora, Gram-negative flora, mammalian flora, animal flora, animal organ flora, feces flora, wastewater treatment flora, brewing flora, water flora, industrial flora, cooling water flora, sewage processing pond flora, plant surface flora, flora involved in industrial processes, food flora, flora associated with food, and combinations of any thereof.

11. A library of sequences representative of a community of organisms produced by a process, the process comprising:
isolating nucleic acid from at least one organism in the community;
contacting the isolated nucleic acid with at least one primer capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate; and
storing the sequence of the amplificate, thus producing the library.

12. The library of claim 11, wherein the at least one primer comprises SEQ ID NO: 1, SEQ ID NO: 2, or SEQ ID NO: 1 and SEQ ID NO: 2.

13. The library of claim 11, wherein sequencing the amplificate comprises sequencing a plurality of amplificates from a plurality of organisms in the community.

14. The library of claim 11, wherein the community of organisms is selected form the group consisting of intestinal flora, vaginal flora, midgut flora, biofilm flora, soil flora, Gram-positive flora, Gram-negative flora, mammalian flora, animal flora, animal organ flora, feces flora, wastewater treatment flora, brewing flora, water flora, industrial flora, cooling water flora, sewage processing pond flora, plant surface flora, flora involved in industrial processes, food flora, flora associated with food, and combinations of any thereof.

15. A process for identifying or enumerating an organism in a community, the process comprising:
generating an organism specific oligonucleotide with a process, the process comprising:
isolating nucleic acid from a normal community;
contacting the isolated nucleic acid with at least one primer capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate; and
designing the organism specific oligonucleotide capable of hybridizing to the sequence of the amplificate;
pooling nucleic acid from a test community;
contacting the pooled nucleic acid with the organism specific oligonucleotide; and
identifying or enumerating the organism in the test community.

16. The process according to claim 15, further comprising amplifying the sequence from the organism.

17. The process according to claim 16, wherein amplifying the sequence from the target organism comprises quantitative amplification.

18. The process according to claim 15, wherein the organism specific oligonucleotide is group-specific.

19. The process according to claim 15, wherein the organism specific oligonucleotide is taxa-specific.

20. The process according to claim 15, further comprising:
tagging the organism specific oligonucleotide with a detectable marker; and
wherein characterizing the organism in the test community comprises detecting the detectable marker.

21. A process for identifying or enumerating an organism in a community, the process comprising:
providing an organism specific oligonucleotide selected from the group consisting of SEQ ID NO: 3, SEQ ID NO: 4, SEQ ID NO: 5, SEQ ID NO: 6, SEQ ID NO: 7, SEQ ID NO: 8, and SEQ ID NO: 9;
pooling nucleic acid from a test community;
contacting the pooled nucleic acid with the organism specific oligonucleotide; and
characterizing the organism in the test community.

22. A process for comparing communities of organisms, the process comprising:
isolating nucleic acid from a first community of organisms;
contacting the isolated nucleic acid with at least one primer capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate;
comparing the sequence of the amplificate to a database of sequences or to the sequence of other amplificates obtained from the first community of organisms, thus generating a profile for the first community of organisms;
isolating nucleic acid from a second community of organisms, wherein the second community of organisms is subjected to conditions different the conditions of the first community;
contacting the isolated nucleic acid from the second community of organisms with the at least one primer capable of hybridizing to the cpn60-like gene;
subjecting the isolated nucleic acid from the second community of organisms and the at least one primer to conditions that allow the at least one primer to amplify a portion of the isolated nucleic acid from the second community of organisms in the presence of a nucleic acid polymerase, thus producing a second amplificate;
sequencing the second amplificate; and
comparing the sequence of the second amplificate to the profile for the first community of organisms.

23. The process according to claim 22, further comprising subjecting the second community of organisms to a stimulus.

24. The process according to claim 23, wherein the first and second communities of organisms comprise a gastrointestinal tract of an animal and the stimulus comprises a change in feed provided to the animal.

25. The process according to claim 22, wherein the condition of the second community comprises a pathology.

26. The process according to claim 22, wherein the second community of organisms originates from a biopsy of a subject.

27. The process according to claim 22, further comprising comparing an abundance of the amplificates of the first community to an abundance of the amplificates of the second community.

28. A process of identifying a taxon having a potential link to a pathology, the process comprising:
obtaining libraries of clones of a target nucleotide sequence;
sequencing the target nucleotide sequence, thus producing nucleotide sequence data;
clustering the nucleotide sequence data based on a phylogenetic analysis;
calculating clone frequencies corresponding to each nucleotide sequence cluster;
creating a frequency ratio between nucleotide sequence clusters; and
comparing the frequency ratio to incidents of the pathology to identify a correlation between the pathology and the frequency ratio.

29. The process according to claim 28, wherein the target nucleotide sequence comprises a portion of a cpn60-like gene.

30. The process according to claim 28, wherein obtaining the libraries of clones of the target nucleotide sequence comprises:
isolating nucleic acid from a community of organisms;
contacting the isolated nucleic acid with at least one primer capable of hybridizing to a cpn60-like gene; and
subjecting the isolated nucleic acid and the at least one primer to conditions that allow the at least one primer to amplify a portion of the isolated nucleic acid in the presence of a nucleic acid polymerase, thus producing the target nucleotide sequence.

31. A process for characterizing a community of organisms, the process comprising:
isolating nucleic acid from the community of organisms;
contacting the isolated nucleic acid with at least one primer, wherein the at least one primer is capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate;
comparing the sequence of the amplificate to a database of sequences or to the sequence of other amplificates obtained from the community of organisms, thus generating a profile of the community of organisms; and
storing the profile of the community of organisms in a database.

32. The process according to claim 31, wherein the community of organisms is selected form the group consisting of intestinal flora, vaginal flora, midgut flora, biofilm flora, soil flora, Gram-positive flora, Gram-negative flora, mammalian flora, animal flora, animal organ flora, feces flora, wastewater treatment flora, brewing flora, water flora, industrial flora, cooling water flora, sewage processing pond flora, plant surface flora, flora involved in industrial processes, food flora, flora associated with food, and combinations of any thereof.

33. The method of claim 31, wherein the profile is stored in an electronic format.

34. A kit for characterizing a community of organisms, comprising:
an oligonucleotide produced by a process, the process comprising:
isolating nucleic acid from a reference community of organisms;
contacting the isolated nucleic acid with at least one primer, wherein the at least one primer is capable of hybridizing to a cpn60-like gene;
subjecting the isolated nucleic acid and the at least one primer to conditions that allow the at least one primer to amplify a portion of the isolated nucleic acid in the presence of a nucleic acid polymerase, thus producing an amplificate;
sequencing the amplificate; and
designing the oligonucleotide capable of hybridizing to the sequence of the amplificate; and
means for amplifying nucleic acid isolated from the community of organisms.

35. A kit for characterizing a community of organisms, comprising:
an oligonucleotide selected from the group consisting of SEQ ID NO: 3, SEQ ID NO: 4, SEQ ID NO: 5, SEQ ID NO: 6, SEQ ID NO: 7, SEQ ID NO: 8, SEQ ID NO: 9, and combinations of any thereof; and
means for amplifying nucleic acid isolated from the community of organisms.

36. A process of characterizing a population of organisms, said process comprising:
identifying a variable region in a nucleotide sequence of interest;
identifying a non-variable region flanking at least one side of the variable region;
obtaining oligonucleotides capable of selectively amplifying the variable region;
obtaining a sample from the population of organisms;
amplifying the variable regions from the sample with the oligonucleotides capable of selecting amplifying the variable region;
sequencing the amplified variable regions;
calculating an abundance of the amplified variable regions;
identifying the origin of the amplified variable regions; and
identifying organisms in the population based on a phylogenetic comparison of the amplified variable regions to a sequence database.

37. The process according to claim 36, further comprising forming a library of the amplified variable regions.

38. The process according to claim 36, further comprising categorizing organisms of the community based on the sequenced amplified variable regions.

39. The process according to claim 38, further comprising comparing an abundance of the categorized organisms from the community to the abundance of organisms of a known community.

40. The process according to claim 36, wherein the nucleotide sequence of interest is one of cpn60, hsp60, groEL, 16s rRNA, 23s rRNA, and the 16s-23s interspacer region.

41. The process according to claim 36, wherein the oligonucleotides are capable of hybridizing to a portion of a cpn60-like gene.
